(19) United States
(12) Patent Application Publication
(10) Pub. No.: US 2004/0067167 A1
(43) Pub. Date: Apr. 8, 2004

(54) Methods and apparatus for optophoretic diagnosis of cells and particles

(75) Inventors: Haichuan Zhang, San Diego, CA (US); Thomas D.Y. Chung, Carlsbad, CA (US); Jeff Hall, Carlsbad, CA (US); William SooHoo, Carlsbad, CA (US); Josh Kohrumel, San Diego, CA (US); Eugene Tu, San Diego, CA (US); Mark Wang, San Diego, CA (US); Daniel Edward Raymond, San Diego, CA (US); Philippe Marchand, Poway, CA (US); Jonathan Diver, San Diego, CA (US); William F. Butler, La Jolla, CA (US); Phan Nguyen, Vista, CA (US); Mirianas Chachisvilis, San Diego, CA (US); Andrew S. Katz, La Jolla, CA (US); Norbert Hagen, Carlsbad, CA (US); Kris Lykstad, San Diego, CA (US); Luis Pestana, San Diego, CA (US)

Correspondence Address: O'MELVENY & MEYERS, 114 PACIFICA, SUITE 100, IRVINE, CA, 92618, US

(73) Assignee: Genoptix, Inc.

(21) Appl. No.: 10/267,914

(22) Filed: Oct. 8, 2002

(51) Int. Cl. G01N 33/48

(52) U.S. Cl. 422/82.05; 422/73

(57) ABSTRACT

A device for characterizing a cell or particle includes a channel having an inlet and an outlet, the channel containing a moving fluid therein for carrying the cell or particle from the inlet to the outlet. The device includes a detector for detecting the presence of a cell or particle along portion of the channel, the detector including a first detecting position, a second detecting position, and a third detecting position. The device further includes a light source providing an optical gradient disposed within the channel and between the second and third detecting positions. A control system is coupled to the detector to receive and process detected signals from the detector. During operation, the amount of time that a cell or particle takes to flow through a first distance (i.e., its time-of-flight) is measured. The cell or particle is then flowed past a second, downstream distance in the presence of an optical gradient and its time-of-flight is measured. A comparison of the measured time-of-flights for the first and second distances is used to characterize the cell or particle. The method can be used to characterize and sort cells based on a biological property.

### RELATED APPLICATIONS

This application is related to application Ser. No. 10/240,611, filed Sep. 12, 2002, entitled “Methods of Using Optical Interrogation to Determine a Biological Property of a Cell or Population of Cells”, which is a continuation-in-part of U.S. application Ser. No. 10/053,507, filed Jan. 17, 2002, entitled “Methods and Apparatus For Generating and Utilizing Linear Moving Optical Gradients,” which itself is a continuation-in-part of U.S. application Ser. No. 09/993,377, filed Nov. 14, 2001, entitled “Methods and Apparatus for Generating and Utilizing a Moving Optical Gradient,” which itself is a continuation-in-part of U.S. application Ser. No. 09/845,245, filed Apr. 27, 2001, entitled “Methods and Apparatus for Use of Optical Forces for Identification, Characterization and/or Sorting of Particles.” This Application is also related to U.S. provisional Application Serial No. 60/377,145, filed on, May 1, 2002, entitled, “Cellular Analysis Using Infrared Moving Optical Gradient Fields”. The above-identified U.S. Applications are incorporated by reference as if set forth fully herein.

### FIELD OF THE INVENTION

The field of the invention relates generally to optical interrogation methods and apparatus used to determine a property of a cell, a population of cells, and/or cellular components, as well as particles. The methods preferably can be used to select, identify, characterize, and sort individual cells, particles, or groups of cells or particles according to the property of interest. The methods can be used in a variety of applications including, for example, drug screening applications, toxicity applications, protein expression applications, rapid clonal selection applications, biopharmaceutical monitoring applications, quality control application, biopharmaceutical enrichment applications, viral detection, bacterial drug sensitivity screening, and environmental testing applications. More particularly, the systems involved may be used to advantageously diagnose the condition or state of a cell or particle.

### BACKGROUND OF THE INVENTION

In the field of biology, there often is a need to discriminate and sort cells or groups of cells based on a particular biological property of interest. For example, the discrimination and separation of cells has numerous applications in pharmaceutical drug discovery, medicine, and biotechnology. As just one example, when cells are used to produce a new protein or biopharmaceutical compound, it is desirable to select those cells or groups of cells that have the highest yield levels. Historically, sorting technologies have utilized some affinity interaction, such as receptor-ligand interactions or reactions with immunologic targets. Sorting technologies using affinity interaction, however, often are labor intensive, costly, require tags or labels, and change the nature or state of the cells.

While biological applications are of particular interest to discriminate and sort cells, similar methods and techniques can be employed in other applications ranging from industrial applications to environmental applications.

Attempts have been made to sort and characterize particles, including cells, based on the electromagnetic response properties of materials. For example, dielectrophoretic separators utilize non-uniform DC or AC electric fields for separation of particles. See, e.g., U.S. Pat. No. 5,814,200, Pethig et al., entitled “Apparatus for Separating By Dielectrophoresis”. The application of dielectrophoresis to cell sorting has been attempted. In Becker (with Gascoyne) et al., PNAS USA, Vol. 92, pp. 860-864, January 1995, Cell Biology, in the article entitled “Separation of Human Breast Cancer Cells from Blood by Differential Dielectric Affinity”, the authors reported that the dielectric properties of diseased cells differed sufficiently to enable separation of the cancer cells from normal blood cells. The system balanced hydrodynamic and dielectrophoretic forces acting on cells within a dielectric affinity column containing a microelectrode array. More sophisticated separation systems have been implemented. Yet others have attempted to use electrostatic forces for separation of particles. See, e.g., Judy et al., U.S. Pat. No. 4,440,638, entitled “Surface Field-Effect Device for Manipulation of Charged Species”, and Washizu “Electrostatic Manipulation of Biological Objects”, Journal of Electrostatics, Vol. 25, No. 1, June 1990, pp. 109-103. Yet others have utilized various microfluidic systems to move and sort particles. See, e.g., Ramsey, U.S. Pat. No. 6,033,546, entitled “Apparatus and Method For Performing Microfluidic Manipulations For Chemical Analysis and Synthesis.”

Still others in the field have used light to sort and trap particles. One of the earliest workers in the field was Arthur Ashkin at Bell Laboratories, who used a laser for manipulating transparent, μm-size latex beads. Ashkin's U.S. Pat. No. 3,808,550 entitled “Apparatuses for Trapping and Accelerating Neutral Particles” disclosed systems for trapping or containing particles through radiation pressure. Lasers generating coherent optical radiation were the preferred source of optical pressure. The use of optical radiation to trap small particles grew within the Ashkin Bell Labs group to the point that ultimately the Nobel Prize was awarded to researchers from that lab, including Steven Chu. See, e.g., Chu, S., “Laser Trapping of Neutral Particles”, Sci. Am., p. 71 (February 1992), Chu, S., “Laser Manipulation of Atoms and Particles”, Science 253, pp. 861-866 (1991).

Generally, the interaction of a focused beam of light with dielectric particles or matter falls into the broad categories of a gradient force and a scattering force. The gradient force tends to pull materials with higher relative dielectric constants toward the areas of highest intensity in the focused beam of light. The scattering force is the result of momentum transfer from the beam of light to the material, and is generally in the same direction as the beam. The use of light to trap particles is also sometimes referred to as an optical tweezer arrangement. Generally, utilizing the Rayleigh approximation, the force of trapping is given by the following equation:

$${F}_{g} = 2 π \cdot {r}^{3} \frac{\sqrt{{ϵ}_{B}}}{c} ( \frac{ϵ - {ϵ}_{B}}{ϵ + 2 {ϵ}_{B}} ) ( ∇ \cdot I )$$

Where F_{g} is the optical gradient force on the particle in the direction toward the higher intensity, r is the radius of the particle, ε_{B} is the dielectric constant of the background medium, ε is the dielectric constant of the particle, I is the light intensity in watts per square centimeter and ∇ is the spatial derivative. FIG. 1 shows a drawing of a particle in an optical tweezer. The optical tweezer consists of a highly focused beam directed to the particle.

As shown in FIG. 1, the focused beam 12 first converges on the particle 10 and then diverges. The intensity pattern 14 relates to the cross-section of the intensity of the beam in the horizontal dimension, and the intensity pattern 16 is the cross-section of intensity in the vertical dimension. As can be seen from the equation, the trapping force is a function of the gradient of the intensity of the light. Thus, the force is greater where the light intensity changes most rapidly, and contrarily, is at a minimum where the light intensity is uniform.

Early stable optical traps levitated particles with a vertical laser beam, balancing the upward scattering force against the downward gravitational force. The gradient force of the light served to keep the particle on the optical axis. See, e.g., Ashkin, “Optical Levitation by Radiation Pressure”, Appl. Phys. Lett., 19(6), pp. 283-285 (1971). In 1986, Ashkin disclosed a trap based upon a highly focused laser beam, as opposed to light propagating along an axis. The highly focused beam results in a small point in space having an extremely high intensity. The extreme focusing causes a large gradient force to pull the dielectric particle toward that point. Under certain conditions, the gradient force overcomes the scattering force, which would otherwise push the particle in the direction of the light out of the focal point. Typically, to realize such a high level of focusing, the laser beam is directed through a high numerical aperture microscope objective. This arrangement serves to enhance the relative contribution from the high numerical aperture illumination but decreases the effect of the scattering force.

Optical trapping methods have been employed to manipulate biological materials. In 1987, Ashkin reported an experimental demonstration of optical trapping and manipulation of biological materials with a single beam gradient force optical trap system. Ashkin, et al., “Optical Trapping and Manipulation of Viruses and Bacteria”, Science, Mar. 20, 1987, Vol. 235, No. 4795, pp. 1517-1520. In U.S. Pat. No. 4,893,886, Ashkin et al., entitled “Non-Destructive Optical Trap for Biological Particles and Method of Doing Same”, reported successful trapping of biological particles in a single beam gradient force optical trap utilizing an infrared light source. The use of an infrared laser emitting coherent light in substantially infrared range of wavelengths, there stated to be 0.8 μm 1.8 μm, was said to permit the biological materials to exhibit normal motility in continued reproductivity even after trapping for several life cycles in a laser power of 160 mW. The term “opticution” has become known in the art to refer to optic radiation killing biological materials.

The use of light to investigate biological materials has been utilized by a number of researchers. Internal cell manipulation in plant cells has been demonstrated. Ashkin, et al., PNAS USA, Vol. 86, 7914-7918 (1989). See also, the summary article by Ashkin, A., “Optical Trapping and Manipulation of Neutral Particles Using Lasers”, PNAS USA, Vol. 94, pp. 4853-4860, May 1997, Physics. Various mechanical and force measurements have been made including the measurement of torsional compliance of bacterial flagella by twisting a bacterium about a tethered flagellum. Block, S., et al., Nature (London), 338, pp. 514-518 (1989). Micromanipulation of particles has been demonstrated. For example, the use of optical tweezers in combination with a microbeam technique of pulsed laser cutting, sometimes also referred to as laser scissors or scalpel, for cutting moving cells and organelles was demonstrated. Seeger, et al., Cytometry, 12, pp. 497-504 (1991). Optical tweezers and scissors have been used in all-optical in vitro fertilization. Tadir, Y., Human Reproduction, 6, pp. 1011-1016 (1991). Various techniques have included the use of “handles” wherein a structure is attached to a biological material to aid in the trapping. See, e.g., Block, Nature (London), 348, pp. 348-352 (1990).

Various measurements have been made of biological systems utilizing optical trapping and interferometric position monitoring with subnanometer resolution. Svoboda, Nature (London), 365, pp. 721-727 (1993). Yet others have proposed feedback based systems in which a tweezer trap is utilized. Molloy, et al., Biophys. J., 68, pp. 2985-3055 (1995).

A number of workers have sought to distort or stretch biological materials. Ashkin in Nature (London), 330 pp. 769-771 (1987), utilized optical tweezers to distort the shape of red blood cells. Multiple optical tweezers have been utilized to form an assay to measure the shape recovery time of red blood cells. Bronkhorst, Biophys. J., 69, pp. 1666-1673 (1995). Kas, et al., has proposed an “optical stretcher” in U.S. Pat. No. 6,067,859 which suggests the use of a tunable laser to trap and deform cells between two counter-propagating beams generated by a laser. The system is utilized to detect single malignant cancer cells. Yet another assay proposed colliding two cells or particles under controlled conditions, termed the OPTCOL for optical collision. See, e.g., Mammer, Chem & Biol., 3, pp. 757,763 (1996).

Yet others have proposed utilizing optical forces to measure a property of an object. See, e.g., Guanming, Lai et al., “Determination of Spring Constant of Laser-Trapped Particle by Self-Mining Interferometry”, Proc. of SPIE, 3921, pp. 197-204 (2000). Yet others have utilized the optical trapping force balanced against a fluidic drag force as a method to calibrate the force of an optical trap. These systems utilize the high degree of dependence on the drag force, particularly Stokes drag force.

Yet others have utilized light intensity patterns for positioning materials. In U.S. Pat. No. 5,245,466, Bums et al., entitled “Optical Matter”, arrays of extended crystalline and non-crystalline structures are created using light beams coupled to microscopic polarizable matter. The polarizable matter adopts the pattern of an applied, patterned light intensity distribution. See also, “Matter Rides on Ripples of Lights”, reporting on the Burns work in New Scientist, Nov. 18, 1989, No. 1691. Yet others have proposed methods for depositing atoms on a substrate utilizing a standing wave optical pattern. The system may be utilized to produce an array of structures by translating the standing wave pattern. See, Celotta et al., U.S. Pat. No. 5,360,764, entitled “Method of Fabricating Laser Controlled Nanolithography”.

Yet others have attempted to cause motion of particles by utilizing light. With a technique termed by its authors as “photophoresis”, Brian Space, et al., utilized a polarized beam to induce rotary motion in molecules to induce translation of the molecules, the desired goal being to form a concentration gradient of the molecules. The technique preferably utilizes propeller shaped molecules, such that the induced rotary motion of the molecules results in translation.

Sasaki et al. discloses a method and device for controlling the flow of fine particles along a pattern formed using a scanning laser. See, Sasaki et al., Pattern Formation and Flow Control of Fine Particles By Laser-Scanning Micromanipulation, Optics Letters, Vol. 16., No. 19 (Oct. 1, 1991). In one demonstration in Sasaki et al., polystyrene latex particles were distributed on in a circular pattern of laser light. A driving force was imparted on the particles by repetitive scanning of the trapping beam at a repetition rate of 15 Hz in a clockwise manner. It was observed that all the particles moved together in an orderly fashion around the circular laser pattern. Experiments were also conducted that varied the repetition rate of the trapping beam. The investigators found that particle flow rates became slower as the scan rate increased.

Various efforts have been described relating to cellular response. By way of example, Ransom et al. U.S. Pat. No 6,280,967 entitled “Cell Flow Apparatus and Method for Real-Time (Sic.) of Cellular Responses” describes an apparatus and method for the real-time measurement of a cellular response of a test compound or series of test compounds on a flowing suspension of cells. The cells and test compound or compounds are combined and then flowed through a detection zone. Typically, a label is detected indicating the response. Libraries of compounds are described. As stated, generally the detectable event requires a label.

In Zborowski et al. U.S. Pat. No. 5,974,901, entitled “Method for Determining Particle Characteristics”, and U.S. Pat. No. 6,082,205, entitled “System and Device For Determining Particle Characteristics”, methods and apparatus are described for determining at least one of a plurality of particle physical characteristics. Particularly, the particle characteristics may include particle size, shape, magnetic susceptibility, magnetic label density, charge separation, dielectric constant, and derivatives thereof. In one aspect, a uniform force field, such as a constant, uniform magnetic force field is generated, the particle is subject to that constant force field, and the velocity determined by observing the particle at multiple locations. Variations are described, such as for determining the position of the particle, though the force field is typically described as being constant. In another aspect, a pre-determined force field magnitude and direction is applied to a particle and multiple digital images are analyzed with specified other components to characterize the particles.

Various researchers have attempted to combine microfabricated devices with optical systems. In “A Microfabricated Device for Sizing and Sorting DNA Molecules”, Chou, et al., PNAS USA, Vol. 96, pp. 11-13, January 1999, Applied Physical Sciences, Biophysics, a microfabricated device is described for sizing and sorting microscopic objects based upon a measurement of fluorescent properties. The paper describes a system for determining the length of DNA by measuring the fluorescent properties, including the amount of intercalated fluorescent dye within the DNA. In “A Microfabricated Fluorescence-Activated Cells Sorter”, Nature Biotechnology, Vol. 17, November 1999, pp. 1109-1111, a “T” microfabricated structure was used for cell sorting. The system utilized a detection window upstream of the “T” intersection and based upon the detected property, would sort particles within the system. A forward sorting system switched fluid flow based upon a detected event. In a reverse sorting mode, the fluid flow was set to route all particles to a waste collection, but upon detection of a collectible event, reversed the fluid flow until the particle was detected a second time, after which the particle was collected. Certain of these systems are described in Quake et al., PCT Publication WO 99/61888, entitled “Microfabricated Cell Sorter”.

Yet others have attempted to characterize biological systems based upon measuring various properties, including electromagnetic radiation related properties. Various efforts to explore dielectric properties of materials, especially biological materials, in the microwave range have been made. See, e.g., Larson et al., U.S. Pat. No. 4,247,815, entitled “Method and Apparatus for Physiologic Facsimile Imaging of Biologic Targets Based on Complex Permittivity Measurements Using Remote Microwave Interrogation”, and PCT Publication WO 99/39190, named inventor Hefti, entitled “Method and Apparatus for Detecting Molecular Binding Events”.

### SUMMARY OF THE INVENTION

A device and associated methods are described for characterizing a cell or particle. The systems generally include a channel having an inlet and an outlet, the channel containing a moving fluid therein for carrying the cell or particle from the inlet to the outlet. The device also includes a detector (or multiple detectors) for detecting the presence of a cell or particle along a portion of the channel. The detector includes at least a first detecting position, a second detecting position, and a third detecting position. The device further includes a light source providing an optical gradient disposed within the channel and between the second and third detecting positions. A control system is coupled to the detector to receive and process detected signals from the detector.

In operation, the amount of time that a cell or particle takes to flow through a first distance (i.e., its time-of-flight) is measured. The particle is then flowed past a second, downstream distance in the presence of an optical gradient and its time-of-flight is measured. A comparison of the measured time-of-flights for the first and second distances is used to characterize the cell or particle. The optical gradient serves as an ‘optical speed-bump’, serving to slightly retard the progress of the cell or particle in an amount related to the degree of interaction between the particle and the optical gradient. In the case of a biological particle such as a cell, the method can be used to characterize cells based on one or more biological properties of the cell. Optionally, the characterization information may be utilized to further sort cells or particles based upon an observed parameter.

A variety of detection systems are described. Within the realm of optical detection systems, coherent light may be used for illumination of the particle. In one embodiment, a pattern generator disposed upstream of the particles selectively illuminates the particle. A detector array determines particle position as a function of time. In an alternative embodiment, a system utilizing coherent light scans a beam over the channel. A detector determines the cell or particle positioning as a function of time. Incoherent light may be used for illumination. Detection may be by any number of techniques, such as through the use of a mask and detector array, or by use of a line camera. Electrical detection of the cell or particle position may be utilized, such as where an impedance detection is utilized.

In a preferred embodiment of the system, an additional detection beam may be utilized. Preferably, the beam is directed axially along the channel and an additional detector is located upstream from the other detectors. By utilizing additional detectors, the various detectors may be optimized to determine different detectable characteristics. The initial detector may be utilized to activate or otherwise tune the remaining detectors. In addition, the initial detector may be used as a gating detector in the sense that it detects the presence of an incoming cell or particle. Preferably the gating detector has the capability to detect whether the incoming test subject (i.e. cell or particle) is in a condition for measurement. For example, if cells are being analyzed on the system, the detector can determine and reject a sample if it appears that the cells are clumped together or otherwise unrepresentative of the cells or particles of interest.

It is an object of this invention to provide a simple, inexpensive, scalable system for Optophoretic diagnostics of a cell or particle. It is a further object of the invention to provide a system that uses low volume, substantially constant velocity flow regulation coupled with optical measurement and interrogation components to serve as a diagnostic device. The device has applications in a wide variety of diagnostic applications including, but not limited to, cancer diagnostic applications and infectious disease diagnostic applications.

### BRIEF DESCRIPTION OF THE DRAWINGS

FIG. 1 is a graphical depiction of optical intensity patterns for a prior art optical tweezer system, showing both the focus beam, a particle and the cross-section of intensity of the beam.

FIG. 2 shows a plan view of a time-of-flight system.

FIG. 3 shows a generalized block diagram of a microfluidic detection system.

FIG. 4 is a block diagrammatic view of one apparatus and associated method for detection, namely one in which coherent light detection is utilized.

FIG. 5A shows a plan diagram of a coherent light detection system utilizing scanning detection.

FIG. 5B graphically shows the operation of scanning system.

FIG. 6A shows a plan view of a system utilizing an incoherent light detection system including a detector mask.

FIG. 6B shows a side plan view of a detector mask.

FIG. 7 shows a plan view of system utilizing incoherent light for detection along with a line camera.

FIG. 8A shows a plan view of an electronic detection system.

FIG. 8B shows close up view of the channel and its associated electrodes.

FIG. 9 shows a side view of a gravity-based time-of-flight system.

FIG. 10 is a plan view of a microfluidic channel based detection system.

FIG. 11 is a system block diagram of various subsystems within the system.

FIG. 12 depicts the optical subsystem for one implementation of a time-of-flight system.

FIG. 13 shows a combined block diagram and processing functionality and software for the acquisition subsystem.

FIG. 14 shows a flow chart of one possible implementation of the software subsystem.

FIG. 15 depicts the forces on a particle in a time-of-flight system.

FIG. 16 depicts the optical force on a typical 10 micron cell by a row numerical aperture (NA) laser line.

FIG. 17 is a graph of time delay as a function of escape velocity normalized to flow velocity for a 10 micron bead as simulated.

FIG. 18 shows the data of FIG. 17 plotted in a log-log format.

FIG. 19 is a depiction of an optical system used to perform line scan and fast scan analysis on samples.

FIG. 20 depicts a preferred detection scheme utilizing multiple detectors and a detection laser.

FIG. 21 illustrates a perspective view of a preferred embodiment of a time-of-flight device.

FIG. 22 illustrates another perspective view of the preferred embodiment of the time-of-flight device shown in FIG. 21.

FIG. 23 schematically illustrates the preferred embodiment shown in FIGS. 21 and 22.

FIG. 24 schematically illustrates the fluidics used in one preferred embodiment of the invention.

FIG. 25 schematically illustrates a preferred embodiment of the flow pump used to create a low, constant flow rate.

FIG. 26 illustrates a preferred embodiment of the flow pump used to create a low, constant flow rate.

FIG. 27 illustrates one embodiment of a massively parallel system.

FIG. 28 illustrates yet another embodiment of a massively parallel system.

FIG. 29A illustrates a device capable of both characterizing and sorting a cell or particle.

FIG. 29B illustrates a preferred device and method for sorting cells or particles in the device shown in FIG. 29A.

FIG. 29C illustrates another preferred device and method for sorting cells or particles in the device shown in FIG. 29A.

FIG. 30 is a histogram of the measured escape velocities of Plasmodium-infected and non-infected red blood cells.

FIG. 31 shows a comparison of the mean escape velocity for Plasmodium-infected and non-infected red blood cells.

FIG. 32 is a histogram of time-of-flight measurements for normal red blood cells and Plasmodium-infected red blood cells.

FIG. 33 shows a comparison of the mean time-of-flight values for the infected and control cells of FIG. 32.

FIG. 34 is a histogram of time-of-flight measurements for normal red blood cells and Plasmodium-infected red blood cells. The cells tested in this experiment were synchronized.

FIG. 35 shows a comparison of the mean time-of-flight values for the infected and control cells of FIG. 34.

FIG. 36 illustrates a histogram of the ratio of T₂/T₁ plotted against the percentage of cancerous and non-cancerous cells from breast tissue.

FIG. 37 shows a comparison of the mean T₂/T₁ ratio of the cancerous and non-cancerous cells of FIG. 36.

FIG. 38 illustrates a histogram of the ratio of T₂/T₁ plotted against the percentage of cancerous skin cells and non-cancerous skin cells.

FIG. 39 shows a comparison of the mean T₂/T₁ ratio of the cancerous and non-cancerous cells of FIG. 38.

FIG. 40 is a scatter plot of the T₂/T₁ ratio as a function of ti for polystyrene beads and PMMA beads.

FIG. 41 is a histogram of the number of particles as a function of T₂/T₁ ratio for experimental data shown in FIG. 40.

FIG. 42 is a scatter plot of the T₂/T₁ ratio as a function of event number for 24657 rho+ (wild type) yeast and MYA-1133 rho(0) (mutant) yeast.

FIG. 43 is a histogram of the percentage of cells as a function of T₂/T₁ ratio for experimental data shown in FIG. 42.

FIG. 44 is a scatter plot of the T₂/T₁ ratio as a function of event number for treated and non-treated HL60 cells. The treated HL60 cells were treated with 1% DMSO.

FIG. 45 is a histogram of the percentage of cells as a function of T₂/T₁ ratio for experimental data shown in FIG. 44.

FIG. 46 is a histogram of the percentage of unactivated and activated T cells as a function of T₂/T₁ ratio.

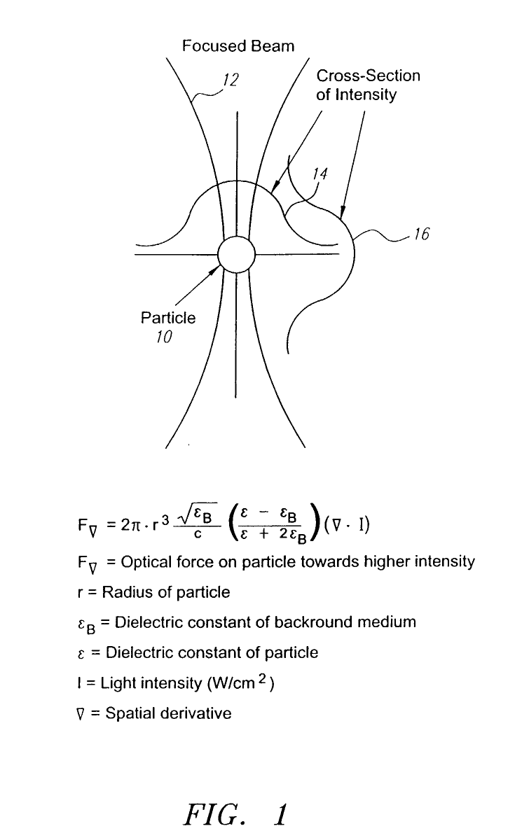

### DETAILED DESCRIPTION OF THE INVENTION

Definitions

The following definitions are provided for an understanding of the invention disclosed herein.

“Biological Property” means a distinct phenotype, state, condition, or response of a cell or group of cells, for example, whether a cell is diseased, has been infected by a virus, the degree to which a cell expresses a particular protein, the stage in the cell cycle a particular cell is presently at, whether the cell is affected by the presence of a chemical compound, a particular phenotype of the cell, whether a ligand is bound to the surface of a cell, cytoskeletal changes in the cell, whether a cell is decorated with antibodies, the presence or absence of a cellular component (e.g., an organelle or inclusion body), a change in one or more cellular components, the toxicity of chemical compounds, a physical property of a cell or population of cells, a response of a cell or population of cells to an external stimulus, cellular motility, membrane fluidity, state of differentiation, viability, size, osmolarity, adhesion, secretion, cell/cell interactions, activation, and cell growth.

“Determining” is meant to indicate that a particular phenotype, state, condition, or response is ascertained.

“Dielectric constant” is defined to be that property which determines the electrostatic energy stored per unit volume for unit potential gradient. (See, e.g., the New IEEE Standard Dictionary Of Electrical And Electronics Terms, © 1993).

The “escape velocity” is defined as the minimum speed at which an interrogated cell or particle no longer tracks the moving optical gradient.

The “optical dielectric constant” is the dielectric constant of a particle or thing at optical wavelengths. Generally, the optical wavelength range is from 150 Å to 30,000 Å.

An “optical gradient field” is an pattern having a variation in one or more parameters including intensity, wavelength or frequency, phase, polarization or other parameters relating to the optical energy. When generated by an interferometer, an optical gradient field or pattern may also be called an optical fringe field or fringe pattern, or variants thereof.

A “moving optical gradient field” is a optical gradient field that moves in space and/or time relative to other components of the system, e.g., particles or objects to be identified, characterized, selected and/or sorted, the medium, typically a fluidic medium, in contact with the particles, and/or any containment or support structure.

An “optical scattering force” is that force applied to a particle or thing caused by a momentum transfer from photons to material irradiated with optical energy.

An “optical gradient force” is one which causes a particle or object to be subject to a force based upon a difference in dielectric constant between the particle and the medium in which it is located.

“Optophoresis” or “Optophoretic” generally relates to the use of photonic or light energy to obtain information about or spatially move or otherwise usefully interact with a particle.

“Optophoretic constant” or “optophoretic signature” or “optophoretic fingerprint” refer to the parameter or parameters which distinguish or characterize particles for optical selection, identification, characterization or sorting.

“Separation” of two objects is the relative spatial distancing over time of a particle from some other reference point or thing.

“Sorting” involves the separation of two or more particles in a meaningful way.

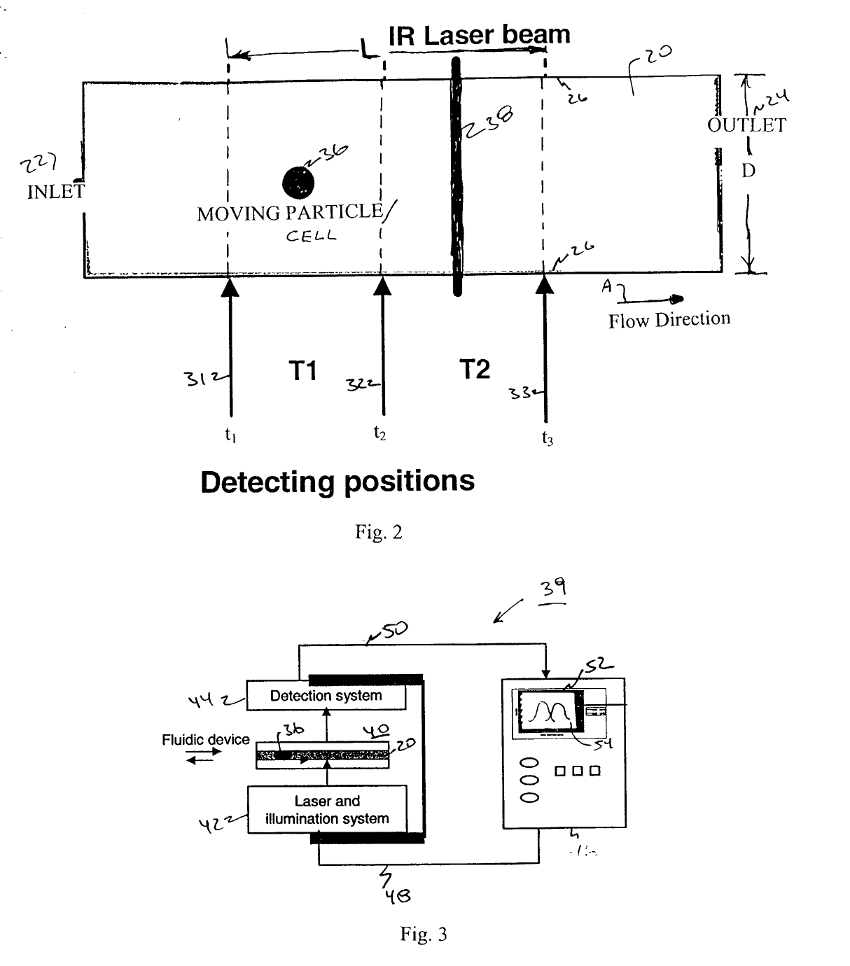

FIG. 2 shows a plan view of a time-of-flight system. A channel 20 is defined by an inlet 22, an outlet 24 and first and second sidewalls 26. Preferably, the sidewalls 26 are linear, and substantially parallel to each other. The outlet 24 has a width D. For biologic diagnostics, such as cell diagnostics, D may be on the order of substantially 60 microns or smaller. Typically, D would exceed the size of the largest expected cell or particle to flow through the channel 20. The channel 20 contains a moving fluid therein, shown by arrow A in FIG. 2, which carries the cells or particles 36 from the inlet 22 to the outlet 24. Particle as used herein refers to any type of small body and includes, as an example, spores, pollen, and particulate matter such as airborne or other environmental contaminants. These include airborne as well as waterborne contaminants. Preferably, the moving fluid flows within the channel 20 at a constant flow rate. The flow rate of the fluid within the channel 20 is preferably controllable. A flow rate is chosen such that the flow rate of the fluid exceeds the escape velocity of the cells or particles 36 transported through the channel 20, that is the flow rate is chosen such that cells or particles 36 do not get “stuck” on the optical gradient 38 (described in detail below).

The system includes multiple, preferably three, detecting positions, 31, 32 and 33. The detecting positions 31, 32, 33, may be associated with detection of times, e.g., t₁, t₂, t₃. The times may be absolute, relative or elapsed. The two outermost detecting positions, i.e., 31 and 33 in this example, are separated by a distance L. A representative length L when detecting biological particles would be substantially 200 microns or less. The difference between the first two detecting positions t₁, t₂, define a time interval T1. T1 may also be associated with a first detection zone corresponding to the motion of the particle 36 through the zone T1. The second and third detecting positions t₂, t₃, define a second detection zone T2. The second detection zone also corresponds to the time difference between t₂, t₃. In one preferred embodiment, the distance between t₂ t₃ is equal to the distance between t₁ t₂.

In operation, the cell or particle 36 flows from the inlet 22 through the channel 20 through the outlet 24. For biological detection purposes, representative particle speeds are from substantially 50 to substantially 200 microns per second. As the cell or particle 36 flows through the first detection zone T1, the time-of-flight through that zone is measured, that is the time it takes for the cell or particle 36 to move from position t₁ to t₂. An optical gradient 38 is disposed within zone T2. Preferably, the optical gradient 38 is substantially linear and has a thickness in the fluid flow direction which is substantially less than the transverse dimension. Preferably, the width is less than substantially 10% of the transverse length of the optical gradient 38. In addition, the optical gradient 38 is disposed within a portion of the channel 20 such that the optical gradient 38 is generally orthogonal to the direction of the fluid flow. The optical gradient 38 is preferably formed using a coherent light source such as a laser that is passed through a cylindrical lens. Alternatively, the optical gradient 38 may be formed using a scanning laser system.

As the moving cell or particle 36 moves through zone T2, it will intercept the optical gradient 38. If the optical gradient 38 has no effect on the moving particle 36, T2 equals T1, assuming the physical difference in detecting positions is equal for zone 1 and for zone 2. If the moving cell or particle 36 does optically interact with the optical gradient 38, the cell or particle 36 will typically be slowed or retarded in its transit through zone T2. Accordingly, T2 would be greater than Ti, assuming the detection positions are uniformly spaced.

The optical gradient 38 may be said to be “static” relative to the underlying device, such as the device that defines the channel 20. However, the relative motion of the cell or particle 36 and the optical gradient 38 provide the discriminating force within the system.

In operation, the cell or particle 36 generally moves at a speed in the range from about 50 to about 200 microns per second. For biological applications, the detection spacing, that is the distance between adjacent detecting positions, is typically on the order from approximately 20 to approximately 50 microns. While shown with three detection positions, the system may use more detection positions, or different types of detectors, as desired. In operation, the throughput of the system may be in the range of approximately 500 to about 2,000 particles per hour per channel 20.

FIG. 3 shows a generalized block diagram of a microfluidic detection, preferably diagnostic, system 39. The substrate 40 containing the channel 20 receives the moving cell or particle 36. An illumination system 42, preferably including a laser as the light source, provides the optical gradient 38. A detection system 44 is operatively positioned to detect the position of the cell or particle 36 at multiple, typically three or more, locations. A control system 46 controls the illumination system 42 via the communication path 48. The output of the detection system 44 is coupled to the control system 46 via the communication path 50. The control system 46 serves to receive and process the detected signals from the detection system 44. A display 54 optionally depicts the detected intensity of the particle as it passes the sections of the detection system 44. In this regard, the display 54 can show the amount of time it takes the cell or particle 36 to pass through zones T1 and T2. The display 54 may further display a ratio using the values of Ti and T2 (i.e. T1/T2 or T2/T1).

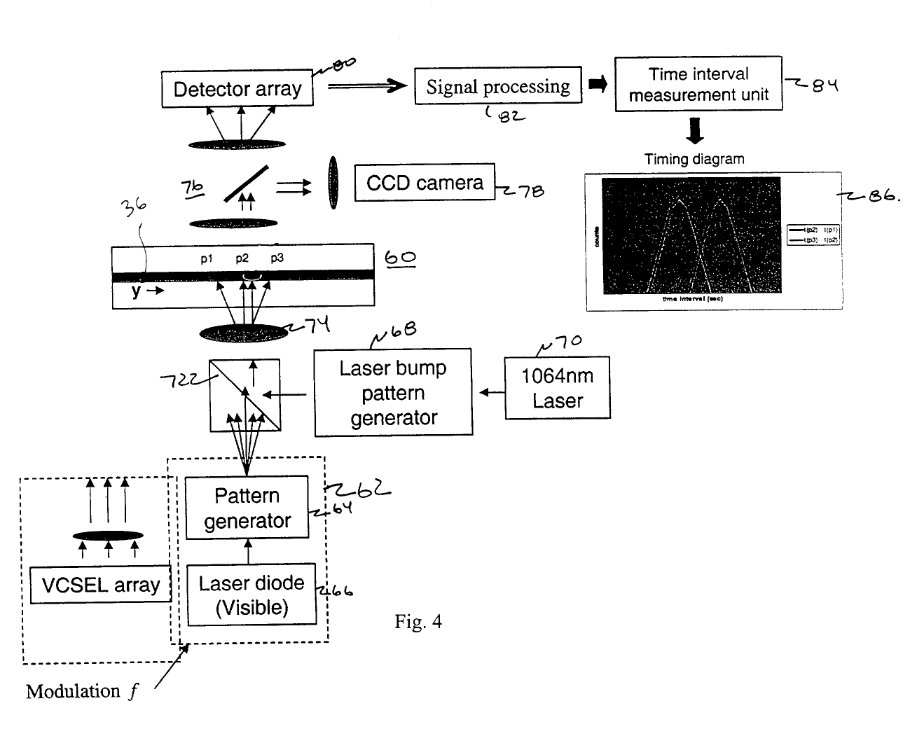

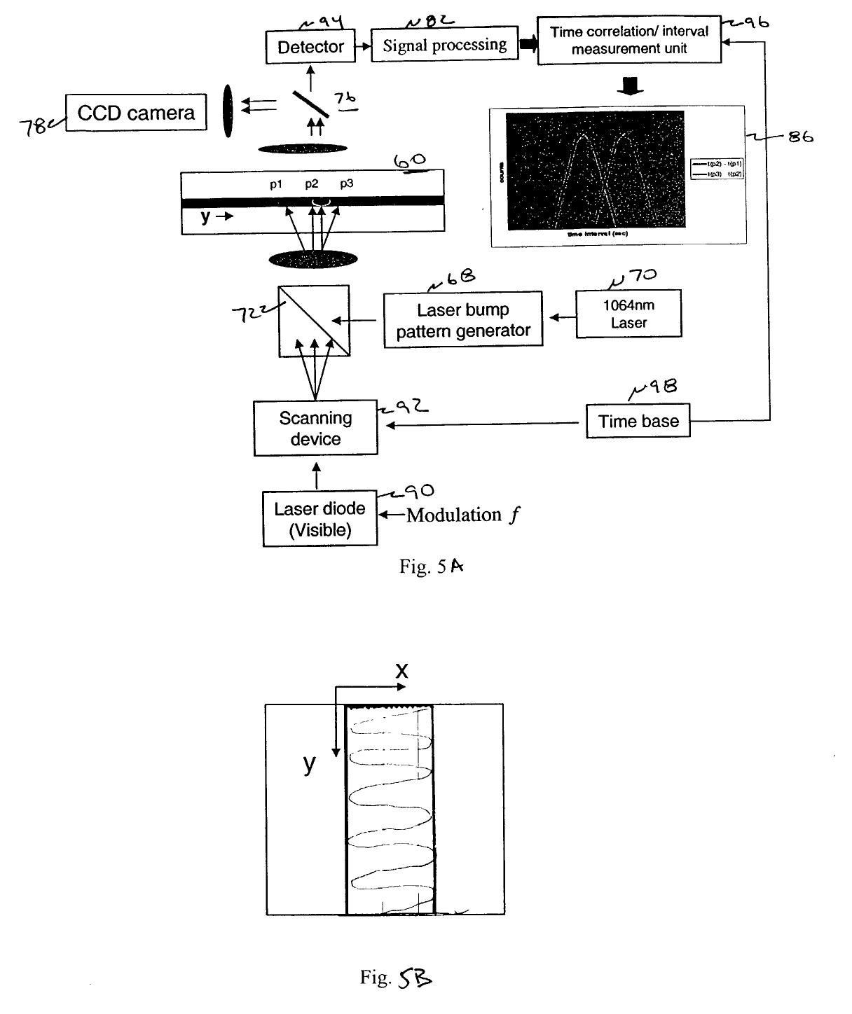

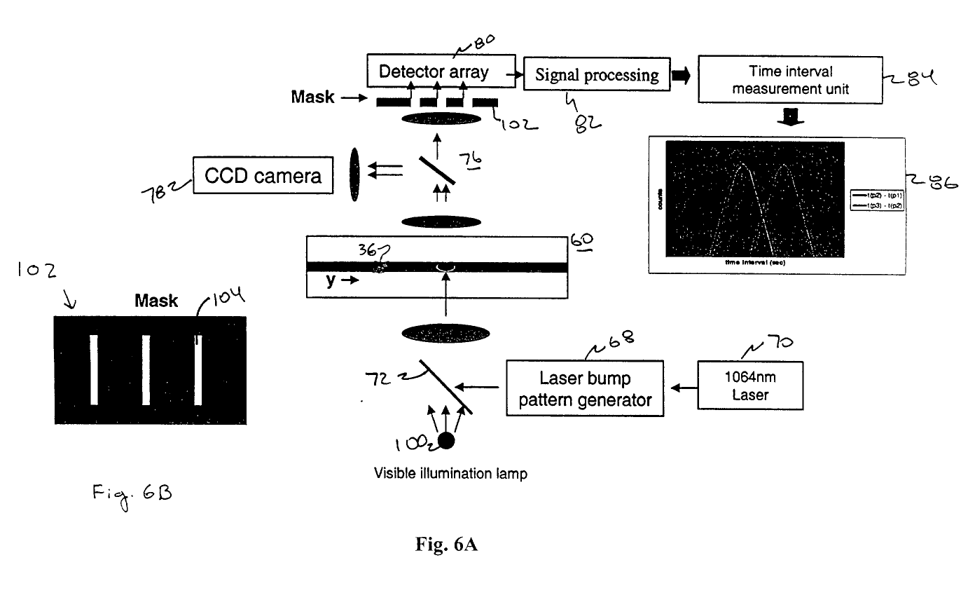

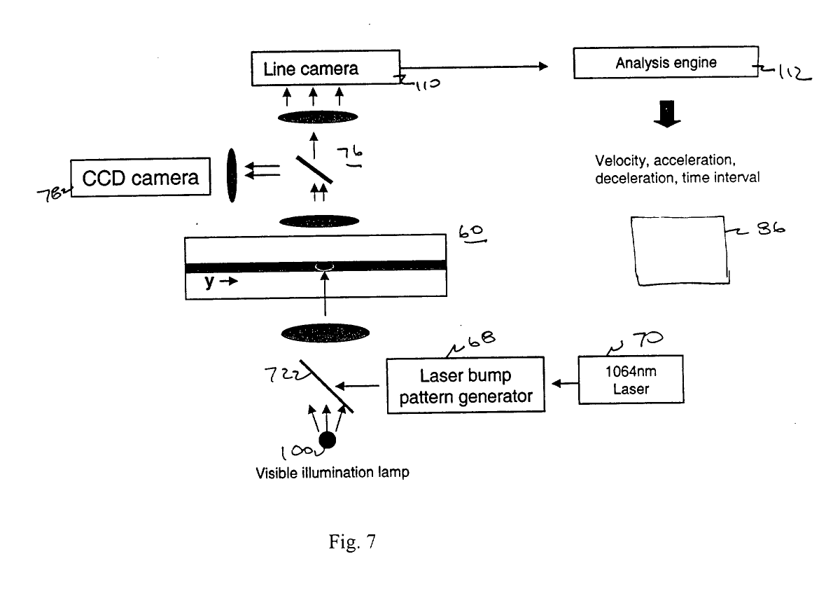

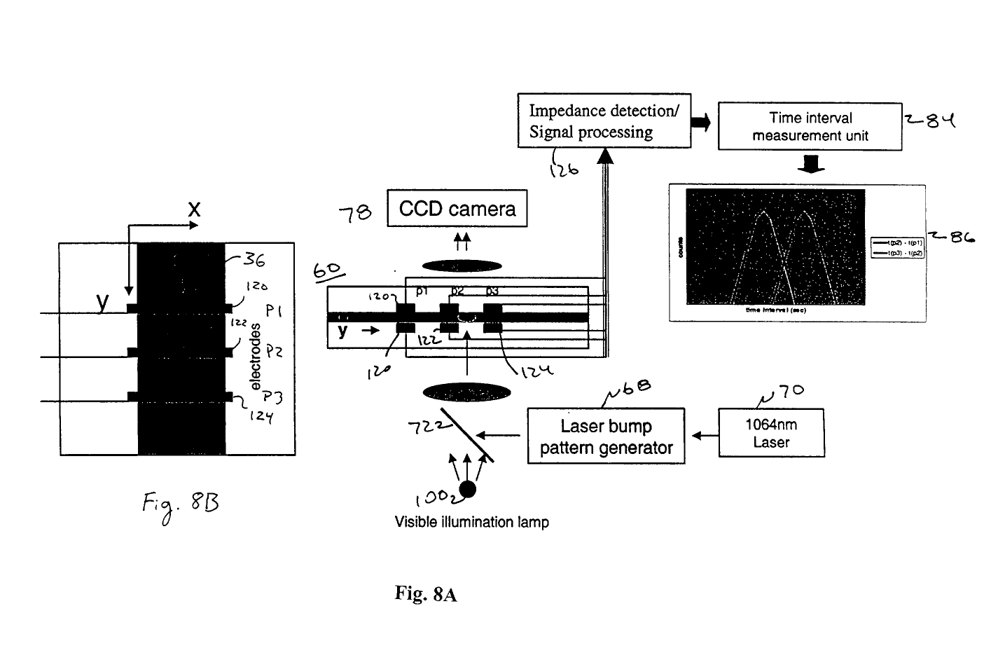

Various detection system may be utilized in connection with such systems. While not meant to be limiting, various exemplary detection systems will be described. FIGS. 4, 5A, 6 and 7 show optical detection systems. More particularly, FIG. 4 is a multi-element detection system utilizing coherent light. FIG. 5A is a scanning detection system using coherent light. FIG. 6 is a detector system using a detector mask, utilizing incoherent light. FIG. 7 is a line camera system utilizing incoherent light. Finally, FIG. 8 shows an electronic detection system, specifically utilizing an impedance detector.

FIG. 4 is a block diagrammatic view of one apparatus and associated method for detection, namely one in which coherent light detection is utilized. A flow cell 60, such as described in connection with FIG. 2 is adapted to receive fluid flow in the direction y, where that fluid includes cells or particles 36 for analysis. An illumination system 62 includes a pattern generator 64 and a light source 66. The pattern generator 64 may be implemented in any number of formats. For example, a VCSEL (vertical cavity surface emitting laser) array may be directed towards the system 60. Alternately, diffractive optical elements may be used. In yet other implementations, light modulators, such as MEMs mirror systems may be utilized. In yet another implementation, light modulator implementation, an AO (Accousto-Optical) modulator may be utilized. However implemented, the illumination system 62 serves to generate patterned illumination for the detection of the cell or particle position, timing or speed. A laser bump pattern generator 68 receives the output of a laser 70 to generate the optical gradient (item 38 in FIG. 2). The pattern generator 68 may be formed from any variety of technologies, e.g., a scanning system, such as an oscillating mirror scanning system, or through the use of diffractive optical elements. As shown, an optical system 72, such as a beam splitter, may be utilized to combine the light from the pattern generator 64 as well as the laser bump pattern generator lens 74. The illumination source 66 may be relatively low powered. For example, the illumination laser diode may be an 8 mW 635 nm laser making the light in the visible range. The laser 70 for generating the optical gradient may be of relatively higher power, such as a 1W laser diode strip. The laser 70 may optionally be in the infrared spectrum, such as 1064 nm. As the particle 36 passes in front of the patterned illumination, modified light passes out from the device 60. Optionally, a beam splitter 76 may be utilized to divert the light to imaging optics which may then be detected with camera 78, such as a CCD camera. A detector array 80 receives the light path from the device 60, after passing through appropriate focusing optics. The detector array may be implemented in any number of technologies. For example, a PIN array may be utilized. The output of the detector array 80 is optionally provided to a signal processor 82, such as a digital signal processor (DSP). A time interval measurement unit 84 detects the time intervals (see, e.g., T1 and T2, or t₁, t₂, and t₃, FIG. 2). A timing diagram 86 may be displayed, such as through a graphical display or a printed display. As shown in FIG. 4, the left most curve depicts the number of counts (i.e., particles 36) in the interval T1, whereas the right most curve depicts the number of counts in the interval T2. As can be seen, the average time to traverse region T2 was greater than the time to traverse region T1.

The camera 78 may be utilized for any functions, including monitoring and alignment. The signal processing system 82 optionally includes demodulation, differential peak detection and digitizing. Optionally, the illumination system 62 may include modulation to enhance the signal to noise ratio (SNR) for detection.

FIG. 5A shows a plan diagram of an coherent light detection system utilizing scanning detection. The description for many components in FIG. 5A is the same as that for FIG. 4, and accordingly those components have been similarly numbered. In FIG. 5A, a coherent light source 90, such as a laser diode, provides illumination of the system 60. The output of the illumination system 90 is scanned over at least a portion of the channel device 60. The scanning device 92 may be, for example, a scanning mirror system, preferably an x, y scanning system such as a system using two rotating mirrors having non-parallel axis. The scanning system would typically scan in a raster scan fashion, such as shown in FIG. 5B. The spacing between the scans is exaggerated in FIG. 5B for purposes of illustration. The output of scanning device 92 is passed through the associated optics to scan the operative portions of the channel device 60. The output of that scan is then imaged upon a detector 94. Optionally, a beam splitter 76 may direct the output illumination to an imaging camera 78, such as a CCD camera. The detector 94 output is then provided to a signal processor 82, which in turn provides its output to an input for the time correlation and interval measurement unit 96. The time correlation and interval measurement unit 96 receives a clock signal input, such as provided from a time base 98. The time base 98 may also control the operation of the scanning device 92. Overall system control may be achieved through such an integrated arrangement.

FIG. 6A shows a plan view of a system utilizing an incoherent light detection system including a detector mask. FIG. 6B shows a side plan view of a detector mask 102. Similar components disclosed in FIGS. 4, 5A, 6A, 7, and 8 are shown having identical element numbers and unless indicated otherwise, operate in the same manner. An illumination source 100, preferably a visible illumination lamp provides a source of light for detection. The light impinges upon the particle to be analyzed, whether passing through focusing optics or not. The light output from the device 60 is then directed to the mask 102. The mask 102 is placed before the detector array 80. As shown, the mask 102 includes three apertures 104. Generally, the apertures are elongate, preferably rectangular, and preferably evenly spaced one from another. The apertures 104 define detection windows for the cell or particle 36 passing through the device 60. In one preferred embodiment, the width of the apertures 104 are about 1.5 μm. Generally, the width of the apertures 104 should be no more than about one-half of the size of the cell or particle 36. In addition, the width of the aperture 104 is chosen such that enough light passes there through to produce a signal strong enough to be picked up by the detector.

FIG. 7 shows a plan view of system utilizing incoherent light for detection along with a line camera. Incoherent light from a source 100 illuminates the cell or particle 36 in the device 60 and the output light is then imaged upon the line camera 110. The output of the line camera 110 is provided to an analysis engine 112. The analysis engine 112 may calculate the desired parameters or properties, such as velocity, acceleration, deceleration, position and/or time intervals. The output of the analysis engine may optionally be displayed on display 86.

FIGS. 8A and 8B show a plan view of an electronic detection system. More particularly, this system utilizes impedance measurement for detection. Detection electrode 120, 122 and 124 are spaced at detection positions P1, P2 and P3, respectively. FIG. 8B shows a plan view of a particle 36 passing through the channel device 60 having three detection electrodes, 120, 122, and 124. An electromagnetic field is set up between the individual electrode pairs, e.g., 120. The impedance of the space between the electrode pairs changes as a function of the particle 36 position. The impedance detector and signal processing system 126 is electrically connected to the electrode pairs, e.g., 120. The profile for the cell or particle 36 as it moves through the detector then is analyzed for the desired property, such as the interval for zones 1 and 2, or the time difference between the two zones.

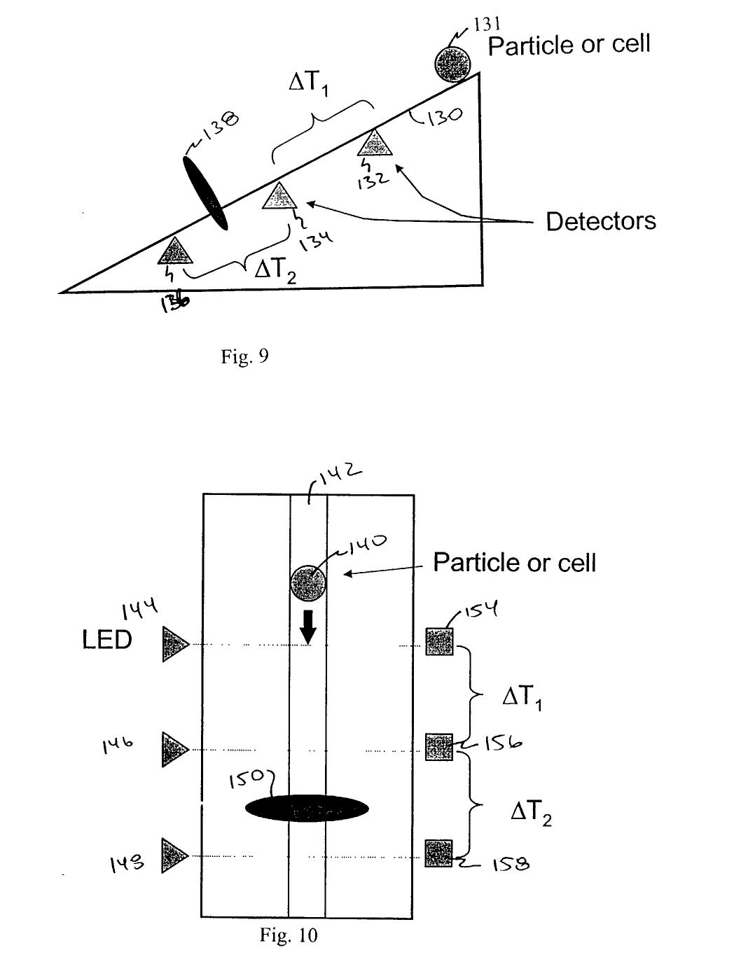

FIG. 9 shows a side view of a gravity-based time-of-flight system. In this regard, an external source of fluid flow such as a pump or the like is not needed. A surface 130 is angled relative to horizontal, such that there is a component of gravitational force exerted on the particle or cell 131 to cause it to move down the surface 130. Detectors 132, 134, 136 are disposed to detect the particle 131 as it passes the detectors. A time difference Δt₁, is detected between the first two detectors 132, 134. An optical gradient 138 comprising an optophoretic “speed bump” is disposed between the second detector 134 and the third detector 136. A time difference is measured between the second detector 134 and the third detector 136, and is designated Δt₂. By analyzing the relative time differences, as described previously, the particle may be characterized.

It should be understood that with respect to the systems and methods described herein that rely an external source of fluid flow such as a pump, the channel 20 (or 142 as described below) may be oriented in any number of orientations including, for example, horizontal and vertical orientations.

FIG. 10 is a plan view of a microfluidic channel based detection system. A particle or cell 140 flows through the channel 142 in the direction of the arrow. A first light source 144, such as a light emitting diode, is directed to a first detector 154, such as a photo detector to detect the time at which the particle or cell 140 reaches a first position. A second light source 146 illuminates the channel 142 and the second detector 156 determines when the particle or cell 140 has reached the second position. Finally, a third source 148 illuminates the channel 142 and third detector 158 detects the crossing of the particle or cell 140 at a third position. An optical gradient 150 provides a potential force against the particle or cell 140. By comparison of the time differences Δt₁, and Δt₂, the particle may be characterized. For cellular applications, the channel would typically be in the range from substantially 10 microns to substantially 100 microns in both width and depth.

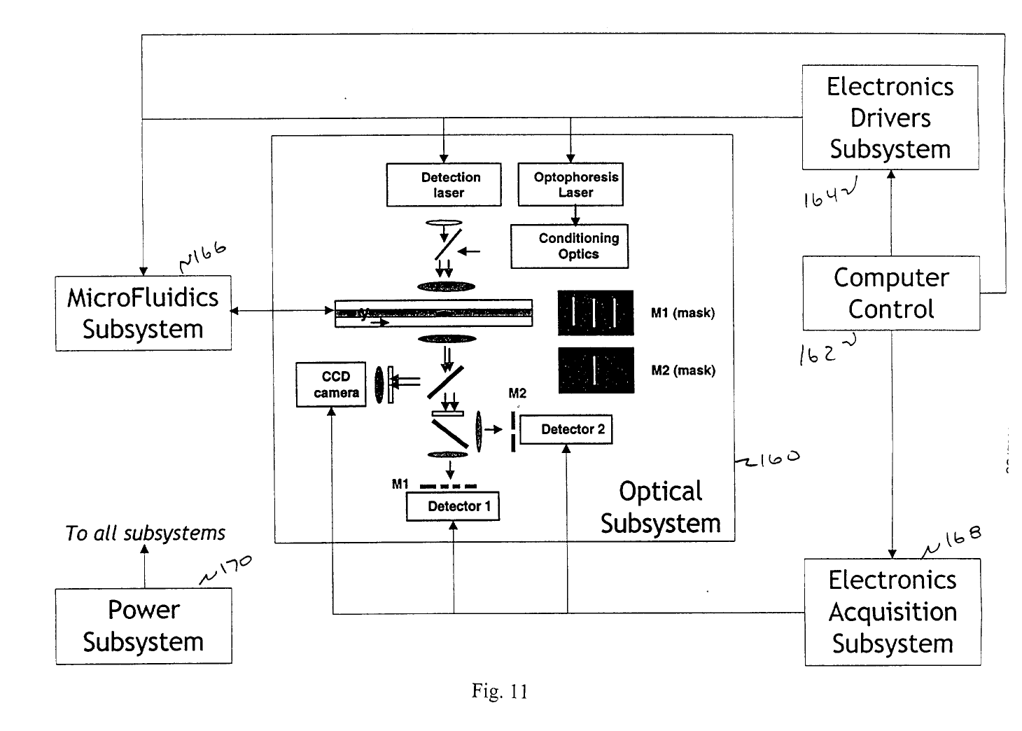

FIG. 11 is a system block diagram of various subsystems within the system. The optical subsystem 160 is depicted using the general structure shown in FIG. 6. However, the description of the system and the various subsystems applies to all of the various methods, especially optical subsystems, described herein. A computer control system 162 interfaces with other subsystems, including the electronics driver subsystem 164, the microfluidics subsystem 166 and the electronics acquisition system 168. As shown, various connections or buses are provided as required between the various subsystems, such as from the computer control subsystem 162 to the microfluidics subsystem 166, and from the electronics driver subsystem 164 to the microfluidics subsystem 166. A power subsystem 170 connects to all subsystems.

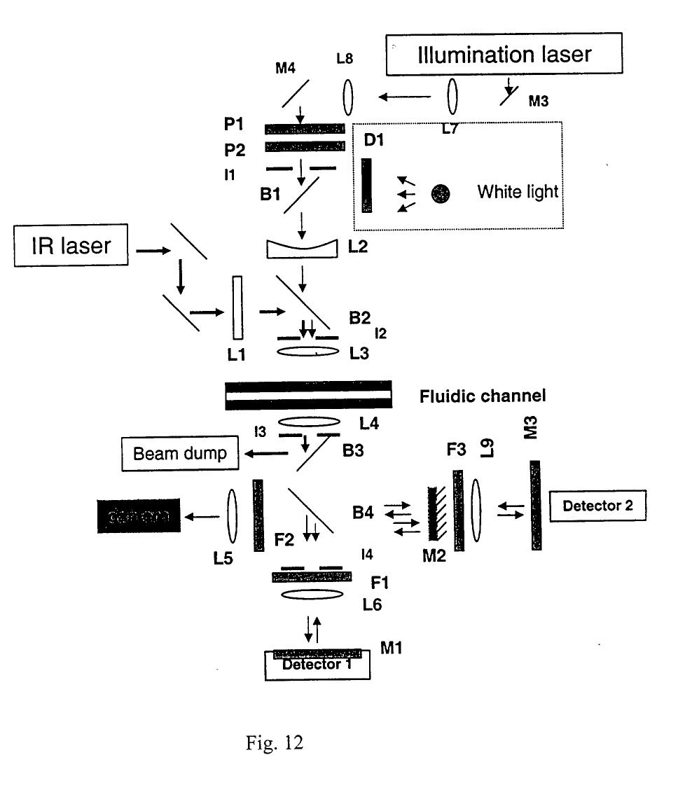

FIG. 12 depicts the optical subsystem for one implementation of a time-of-flight system.

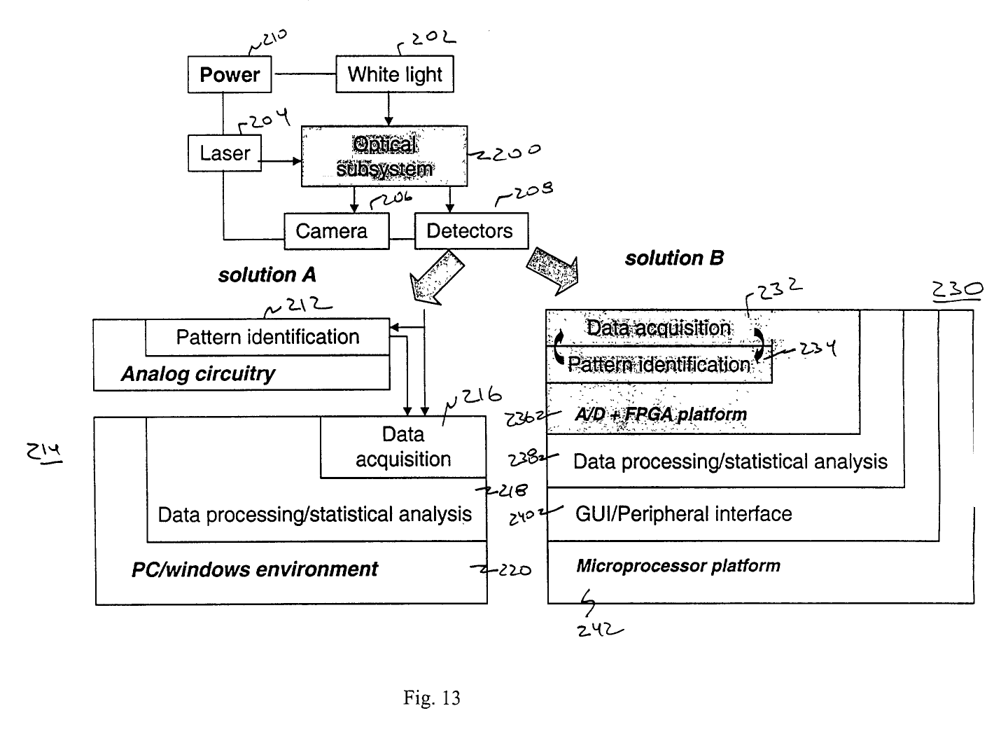

FIG. 13 shows a combined block diagram and processing functionality and software for the acquisition subsystem. An optical subsystem 200 receives optical input from a white light source 202 and a laser 204 to generate the optical gradient. Detection may consist of detectors 208 to detect the time of crossing of the particle at a predefined position. An imaging camera 206 may be utilized for general system imaging, alignment or to otherwise determine the location of the particle. A power system 210 is connected to all necessary electrical components. FIG. 13 shows two possible detection, analysis and display systems. Under solution A, a pattern identification step 212 may be utilized. Analog circuitry may be implemented or various digital techniques, such as digital signal processors (DSPs) may be utilized. The analysis section 214 receives the output of the pattern identification 212 and optionally the direct output of the detector 208, which is received by the data acquisition subsystem 216. A data processing system 218 performs various functions, including optionally statistical analysis. The various functionalities of system 214 may be performed under control of a personal computer, such as operating in a windows based environment.

Solution B depicts the functionality 230 in which the output of the detectors 208 is provided to a data acquisition functionality 232 and/or a pattern identification subsystem 234. If the data acquisition functionality 232 and pattern identification functionality 234 are present, they may be performed in either order. As depicted, an analog-to-digital (A/D) converter 236 is provided to convert acquired analog data to digital data. Optionally, an FPGA platform may be utilized. The acquired data is then subject to data processing step 238, which optionally includes statistical analysis. Ultimate display to the user may be under control of a graphical user interface or peripheral interface 240. In this embodiment, the system may be under microprocessor control 240.

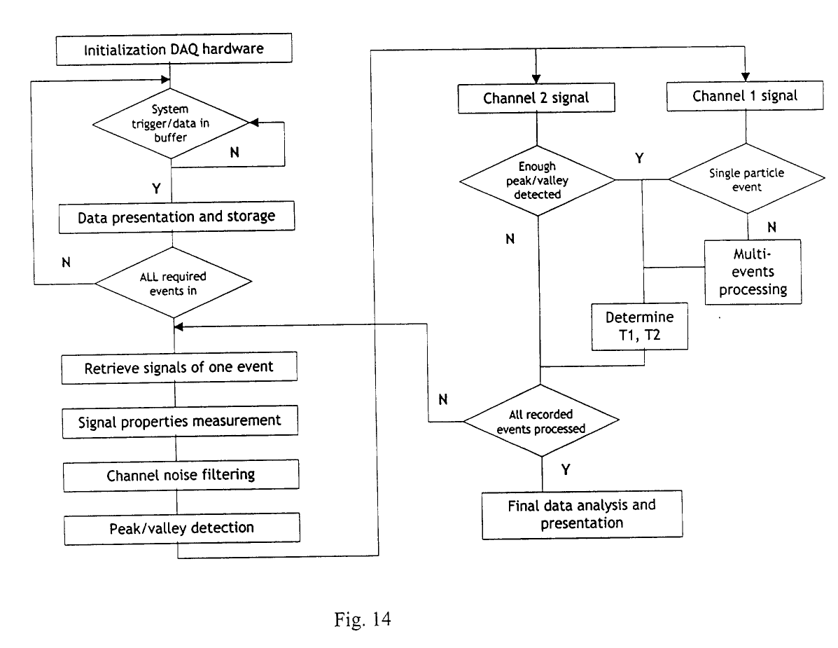

FIG. 14 shows a flow chart of one possible implementation of the software subsystem.

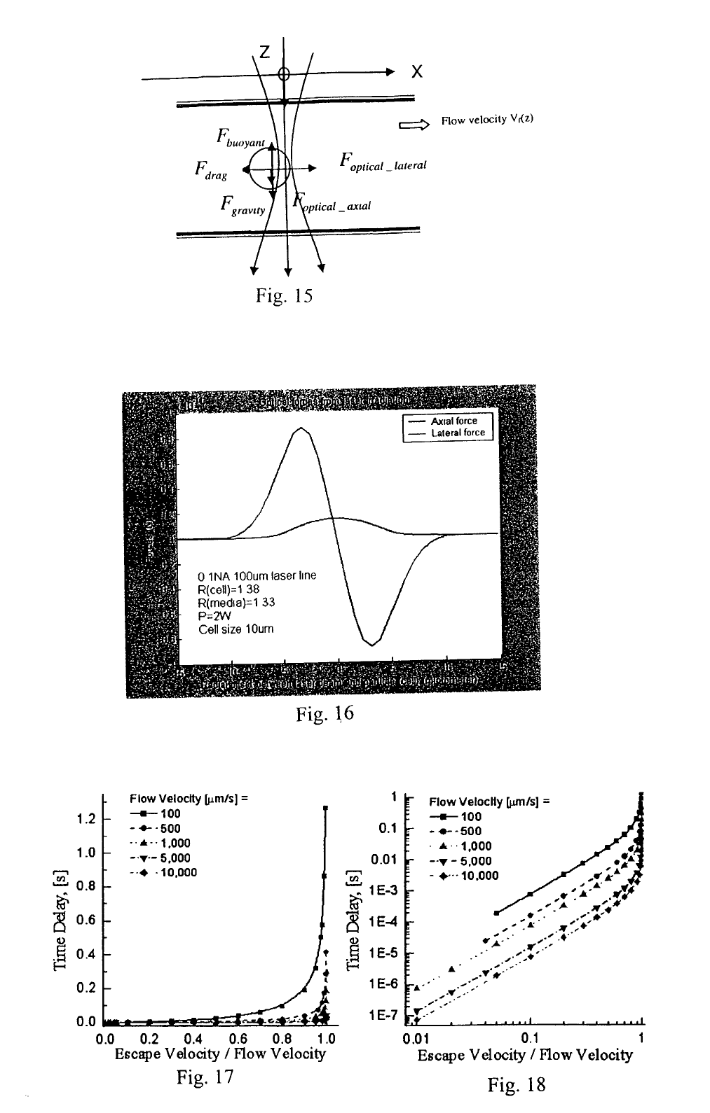

FIG. 15 depicts the forces on a cell or particle in a time-of-flight system. TOF (Time-of-flight) system measures the time delay of flowing particles or cells by a laser beam in micro fluidic environment. The time delay according to optophoretic property is used to analyze the biological differences between populations. The TOF instrument is aiming for low cost and diagnostic applications. For a particle or cell flowing thought an optical field inside a micro-channel, the forces applied are shown in FIG. 15. The force equation is:

$$m \frac{{ⅆ}^{2} x}{ⅆ {t}^{2}} = {F}_{optical\_lateral} ( x ) - {F}_{drag} ( \frac{ⅆ x}{ⅆ t} - {v}_{f} ( z ) , z ) \qquad Equation 1$$

Assume: z=z₀ constant before particle interacts with laser beam (z₀ is the middle of the channel), and gravity force is balanced with buoyant force. Then:

$${F}_{drag} ( \frac{ⅆ x}{ⅆ t} - {v}_{f} ( {z}_{0} ) , {z}_{0} ) = b ( \frac{ⅆ x}{ⅆ t} - {v}_{f} ) \qquad Equation 2$$

where b is the drag coefficient that depends on the radius and shape of the object and the viscosity (“stiffness”) of the medium. For a sphere of radius r, the drag coefficient is

b=6π·rη Equation 3

where η is the viscosity of medium in g/cm s. Due the boundary effect, the flow velocity decreases upon the distance to the boundary of the micro-channel. Both GLMT and geometrical simulations find that optical forces F_{optical}_{_{—}}ₗₐₜₑᵣₐₗ(x) and F_{optical}_{_{—}}ₗₐₜₑᵣₐₗ(x) on a cell by low numerical aperture (NA) laser beam are typically as shown in FIG. 16. FIG. 16 depicts the optical force on a typical 10 micron cell by a 0.1 (NA) laser line.

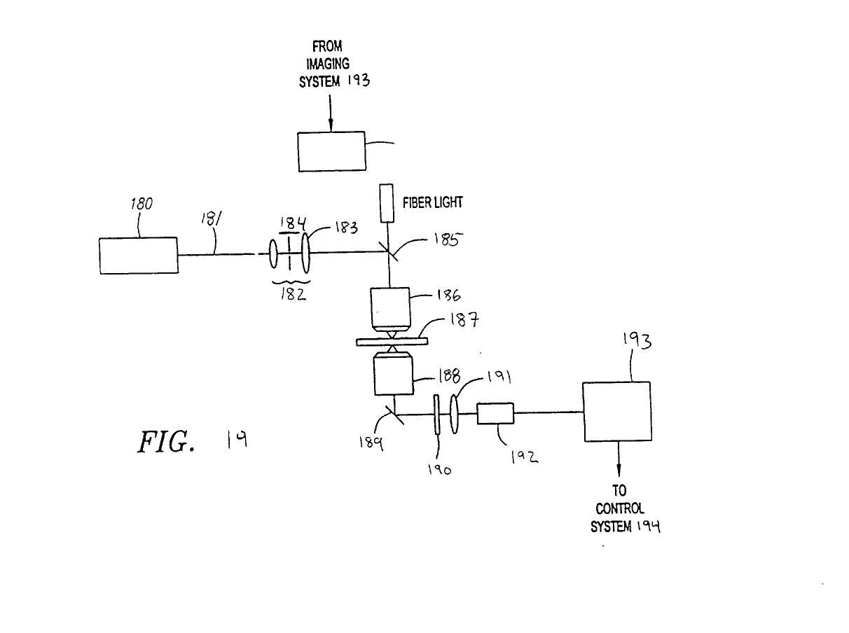

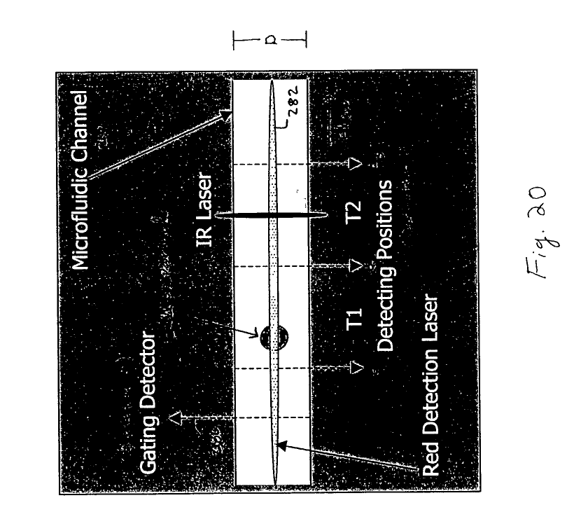

Simulations have been performed since Equation 1 is difficult to solve because F_{optical}_{_{—}}ₗₐₜₑᵣₐₗ(x) is in nonlinear and in complicated form. Several numerical simulations have been conducted as shown in FIGS. 17 and 18. These simulations have not considered the impact from the axial optical force.

FIG. 17 is a graph of time delay as a function of escape velocity normalized to flow velocity for a 10 micron bead as simulated. As expected, as the flow velocity approaches the escape velocity, the time of delay increases. In addition, FIG. 17 shows that lower flow velocities produce larger time delays for a given escape velocity/flow velocity. FIG. 18 shows the data of FIG. 17 plotted in a log-log format. The devices and methods described herein may take advantage of the interaction between flow velocity and delay time to optimize the sensitivity of the device. For example, as seen in FIG. 17, the time delay increases as the flow velocity approaches the particle's escape velocity. This fact may be exploited by modifying the flow velocity to a level just above the escape velocity of the particle to thereby create a large degree of time delay. In addition, FIG. 17 shows that the slower flow velocities produce the largest time delays. Consequently, slower fluid flow rates will produce more noticeable changes in the measured travel times in the detection zones (i.e., zone T2 as compared to T1). Of course, slower flow rates will reduce the overall throughput of the device and may not be desirable in certain applications.

Regardless the accuracy and parameters of the simulations, the non-linearity of TOF system is clear. These simulations are supported by experimental results from fast scan instruments, which have strong similarities to the TOF system. The TOF system is sensitive to optophoretic differences in biological cells. The biological sensitivities of TOF system have been demonstrated.

FIG. 20 shows a preferred signal detection scheme utilizing multiple detectors. FIG. 20 includes components similar to those described in FIG. 2. FIG. 20 includes an additional detection position 280. A detection laser beam 282 is directed to the system in a direction generally parallel to the channel 20. Preferably, the detection laser beam 282 is a low power visible laser beam. The detectors may be any of the type described generally herein. The added detector may be optimized for properties other than that which the other particle detectors are optimized for. For example, the detectors may be optimized based on optical arrangement, such as optical focusing, and/or the geometry of the detector mask, and/or by electronic processing, such as by dedicated filtering and/or the use of threshold circuits. In this way, the signal from the additional detector may be more representative of particle physical properties, such as particle physical size. In contrast, the first detectors may be optimized for detecting the particle physical position. In a preferred embodiment, the additional detector may be physically placed upstream of the other detection positions. In this arrangement, the additional detector may provid trigger selection for the time-of-flight signal acquisition based upon a particles detected property or properties. In this regard, the additional detector acts as a gating detector that indicates to the other components of the device, i.e., the detectors that measure the time intervals T1 and T2 that a cell or particle is about to pass through the detection zones. Throughput and accuracy of signal acquisition generally increases by adding an additional detector. Optionally, yet additional detectors having different optical and/or electrical arrangements may provide more information regarding the particle measurement. Such additional properties or parameters might include, e.g., light scatter, absorption by the particle, size, autofluorescence, fluorescence, luminescence, and other reporter-based properties.

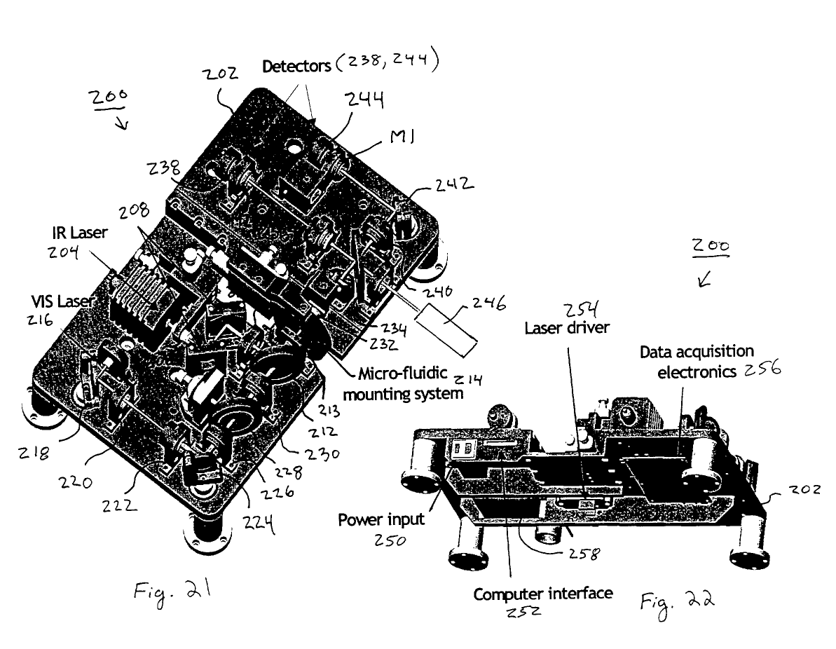

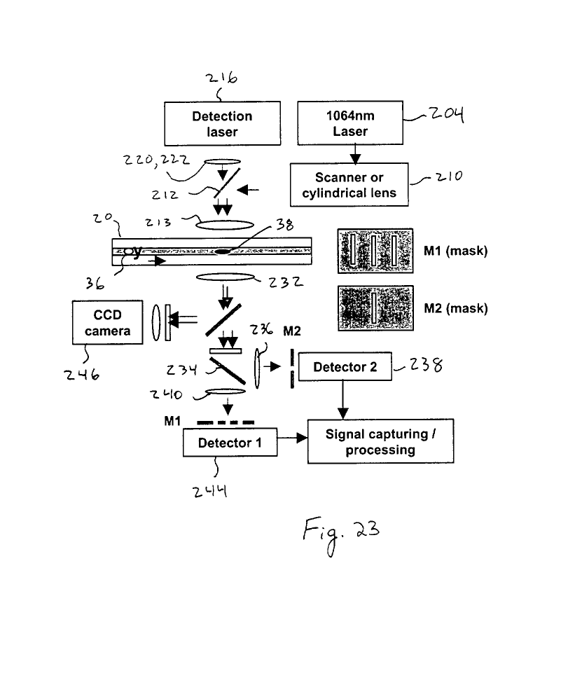

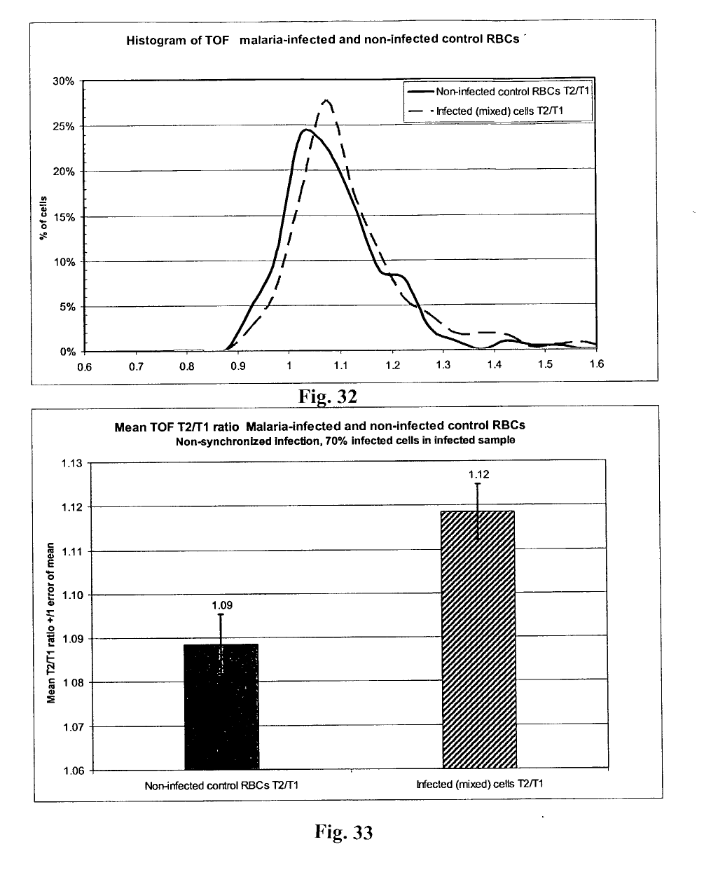

FIGS. 21 and 22 show a preferred embodiment of a time-of-flight device 200 that employs an additional detector as is described above. The device 200 includes optical, mechanical, and fluidic components that are mounted on a base plate 202. With reference to FIG. 32, the device 200 includes a infrared (IR) laser 204 that outputs a coherent beam of infrared light. Preferably, the IR laser 204 has a wavelength in the range of about 780 nm to about 1064 nm. Two preferred wavelengths within this range include 808 nm and 1064 nm. The IR laser 204 creates the optical gradient (e.g., optical gradient 38 as is shown in FIG. 2) that is used to differentially slow cells or particles passing there through. The output of the IR laser 204 passes through a laser collimation lens 206 and is directed against two mirrors 208. The light then passes through a IR cylindrical lens 210 and into an IR beam splitter 212. The light passing through the IR beam splitter 212 is then directed through a focusing lens 213 into a microfluidic mounting system 214. The microfluidic mounting system 214 includes at least one channel (not shown) therein through which the cells or particles pass. The IR optical gradient is disposed generally perpendicular to the direction of flow through the channel contained in the microfluidic mounting system 214.

The time-of-flight device 200 also includes a visible (VIS) laser 216 that outputs a coherent beam of light. Preferably, the visible laser 216 comprises a laser diode operating at around 635 nm. The visible laser beam acts as a detection laser as is described in more detail above and as shown in FIG. 20. The visible laser beam is reflected off another mirror 218 and passed through two lenses 220, 222. The visible laser beam is then reflected off a mirror 224 and passed through a filter 226. The visible laser beam then passes through a white light beam splitter 228 and into a cylindrical lens 230. The visible laser beam then passes through the focusing lens 213 and into the microfluidic mounting system 214. The visible laser beam produces a line of light that is disposed within the channel (not shown) contained within the microfluidic mounting system 214 in a direction that is generally parallel to the direction of fluid flow. This detection laser is shown in detail in FIG. 20 (detection laser 282).

After passing through the microfluidic mounting system 214, both the IR laser beam and the visible laser beam pass through a collection lens 232 and into a beam splitter 234. One output of the beam splitter 234 is then directed through an imaging lens and filter 236. The light passing through the imaging lens and filter 236 is then directed through a mask M2 disposed in front of a first detector 238. The mask M2 preferably includes a single window therein as is shown, for example, in FIG. 11. The first detector 238 is preferably used as an event or gating detector as is described above and shown in FIG. 20.

The other output of the beam splitter 234 is directed to an imaging lens and filter 240 and into a mirror 242. The reflected light is then directed through a mask M1 disposed in front of a second detector 244. The mask M1 preferably includes three windows therein as is shown, for example, in FIG. 11. The second detector 244 is preferably used to determine the amount of time it takes a cell or particle to travel from a first detecting position t₁ to a second detecting position t₂ and the amount of time it takes the same cell or particle to travel from the second detecting position t₂ to a third detecting position t₃. The three windows in the mask M1 correspond to the detecting positions t₁, t₂, and t₃.

The device 200 may also include an optional camera 246 such as a CCD camera that is used to image the microfluidic mounting system 214. The optional camera is used to calibrate the device 200. If the optional camera 246 is used, a white light source 248 is preferably used to provide additional light to enhance the imaging of the microfluidic mounting system 214 and the channel contained therein.

FIG. 33 illustrates the underside of the device 200. The device 200 includes a power input 250 that is preferably connected to a conventional 110 VAC power source. The device 200 also includes a computer interface 252 that allows data communication between the device 200 and an external computer (not shown). Preferably, the computer interface 252 is a 68 pin high-speed connection. FIG. 33 also illustrates the laser driver 254 for the IR laser 204. Data acquisition electronics 256 are included in a printed circuit board preferably located on the underside of the device 200. The data acquisition electronics 256 includes therein a driver for the visible laser 216. A power supply 258 for the device 200 is located on the underside of the base plate 202.

FIG. 23 schematically illustrates the preferred embodiment illustrated in FIGS. 21 and 22 along with the channel 20 of the microfluidic mounting system 214. The optical gradient 38 is shown disposed inside the channel 20. A cell or particle 36 travels down the channel 20 in the direction of the arrow. Detectors 238 and 244 are coupled to signal capturing/data processing electronics 260.

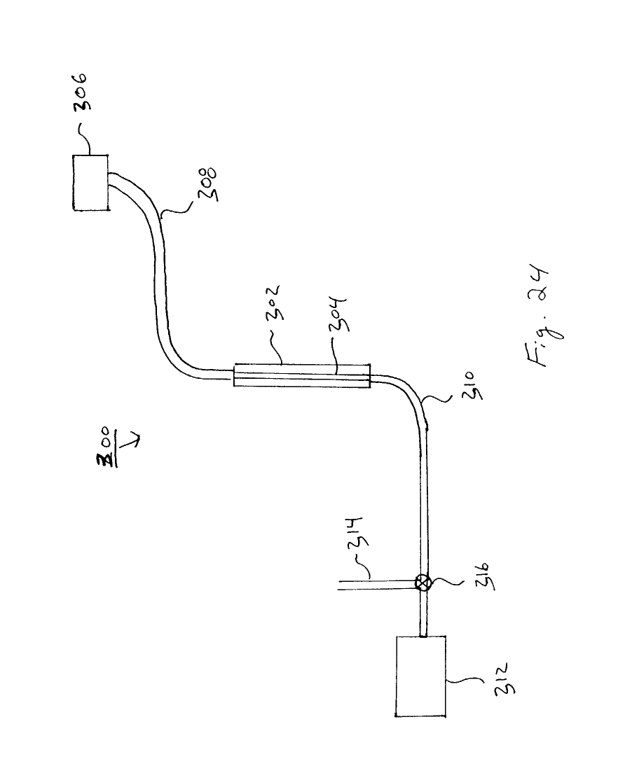

FIG. 24 schematically illustrates the fluidics 300 used according to one preferred embodiment to produce a substantially constant, low flow rate through a device 302 containing a channel 304 therein. In FIG. 23 the channel 304 is oriented in the vertical direction. A reservoir 306 is provided that contains the cells or particles in a fluid medium. Tubing 308 is provided between the reservoir and the inlet to the channel 304. Additional tubing 310 is provided at the outlet of the channel 310 and connects to a flow pump 312. The flow pump 312 is used to provide a substantially constant yet low flow rate of fluid through the channel 304. The flow pump 312 is advantageously controllable so that various flow rates can be used. Also preferably included in the fluidics is a bypass 314. The bypass 314 is used to evacuate fluid from the system after passing through the device 302. A controllable valve 316 is shown in FIG. 23 that is used to direct fluid from the flow pump 312 to the bypass 314.

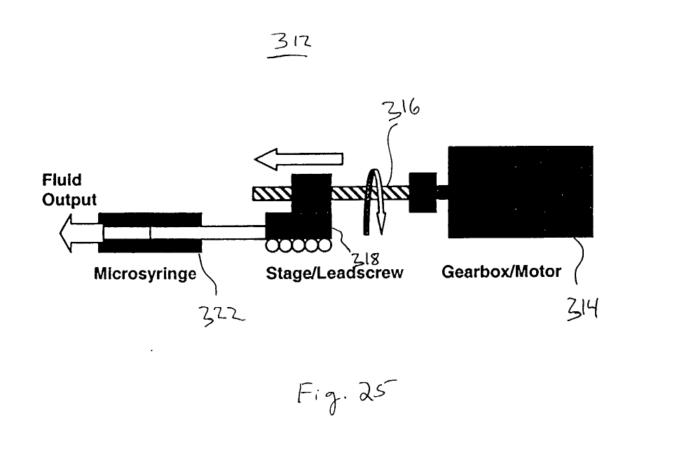

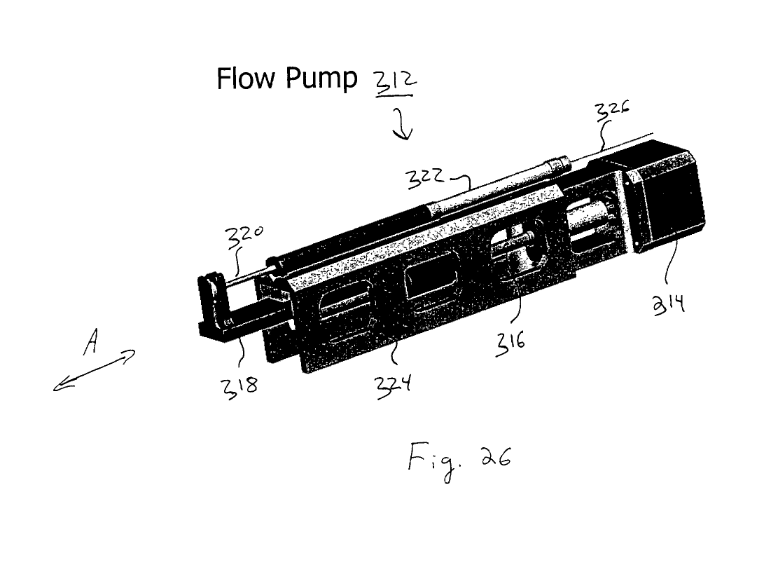

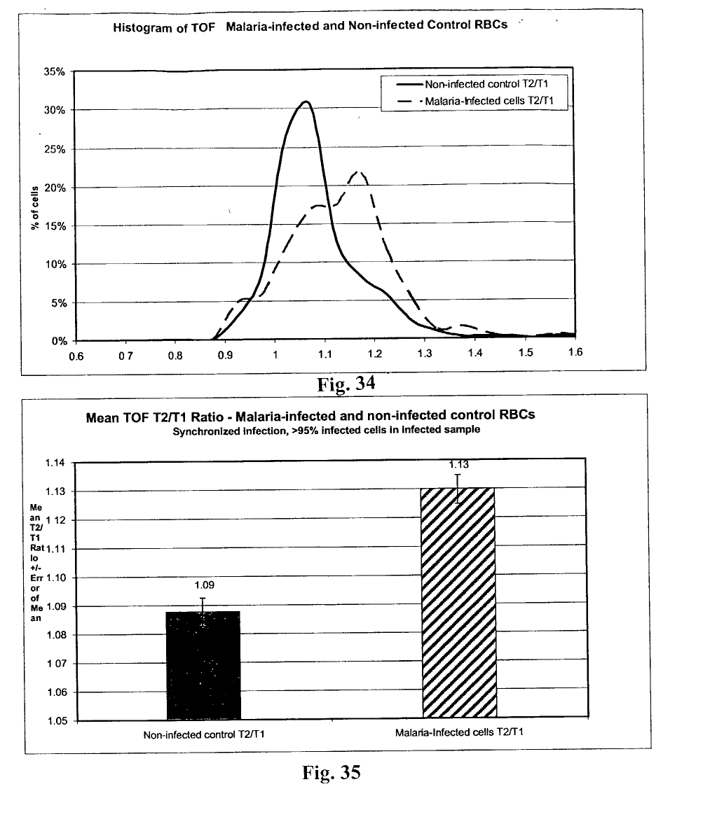

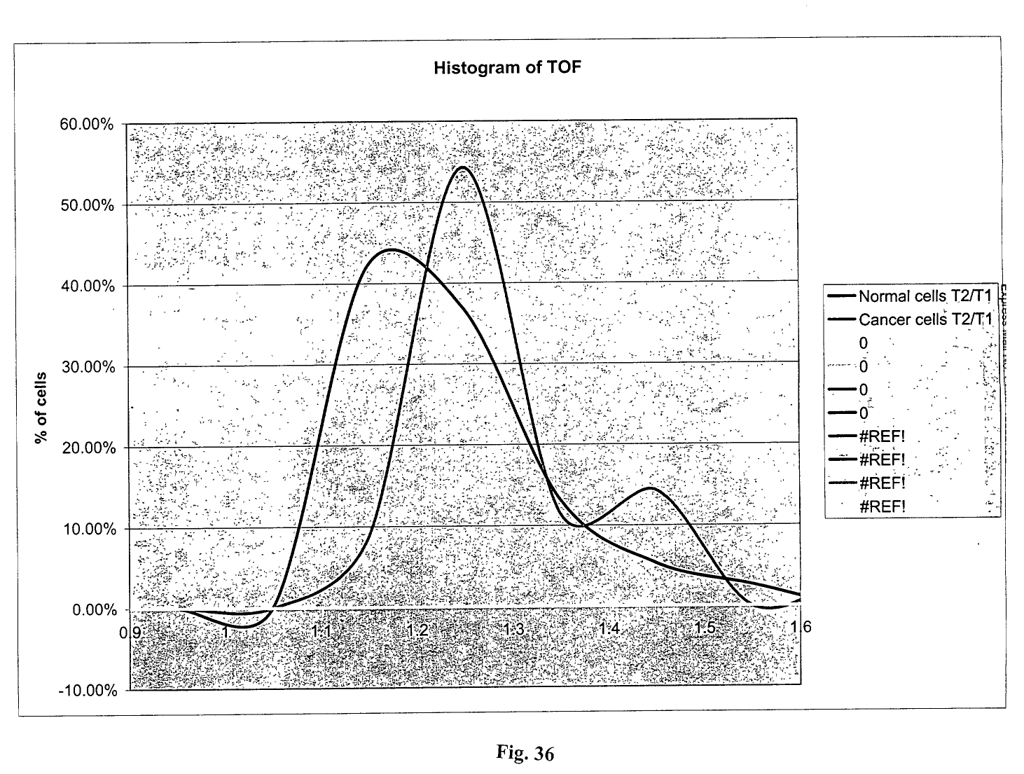

FIGS. 25 and 26 illustrate a preferred embodiment of the flow pump 312. In this embodiment, the flow pump 312 includes a motor 314. The motor 314 is preferably a stepper motor with an integral 50× to 100× gear reduction drive of the output rotation rate. The motor 314 is coupled to rotationally drive a leadscrew 316. The leadscrew 316 preferably is a high pitch leadscrew. The leadscrew 316 is mechanically coupled to a stage 318. Rotation of the leadscrew 316 imparts linear motion to the stage 318 in the direction of arrow A shown in FIG. 36. As best seen in FIG. 36, the stage 318 is coupled to a syringe plunger 320. A stationary syringe 322 is mounted atop a housing 324. Preferably the syringe 322 is a microsyringe with a volume of between about 1 μL to about 100 μL. Preferably the plunger 320 has a travel distance (stroke) of between about 2 to about 5 cm. It is generally preferably to use a syringe 322 with a small cross-sectional area and a long stroke. These two conditions advantageously produce low flow rates. Tubing 326 is provided at the end of the syringe 322 that is opposite to the end of the syringe 322 with the plunger 320. The tubing 326 is, in turn, connected to a device such as device 302 shown in FIG. 34.

During operation, the motor 314 rotates the leadscrew 316 which causes the stage 318 and its connected plunger 320 to move in the axial direction (Arrow A in FIG. 36). In one preferred embodiment, the syringe 322 is used to withdraw fluid containing cells or particles from a reservoir such as reservoir 306 shown in FIG. 34. The cells or particles then pass with the fluid through a channel such as channel 304 shown in FIG. 34. After the plunger 320 has traveled its maximum distance, the plunger 320 can be depressed into the syringe 322 using the motor 314 operating in the reverse direction to evacuate the fluid and cells/particles contained therein using, for example, the bypass 314. In an alternative embodiment, the syringe 322 may be preloaded with cells or particles and the fluid can then be pushed through the channel 304 by depressing the plunger 320 using the motor 314.

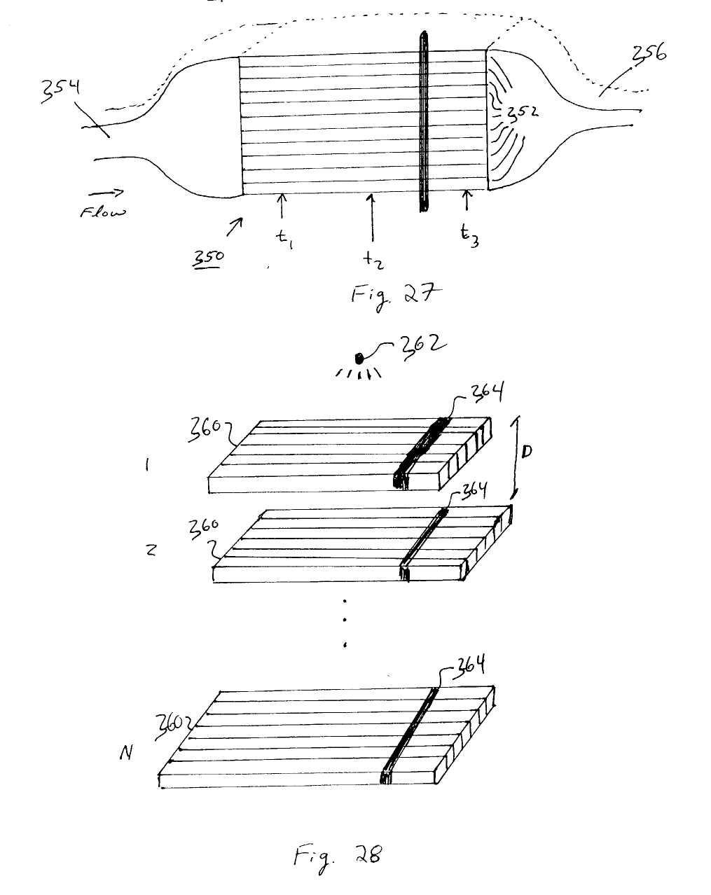

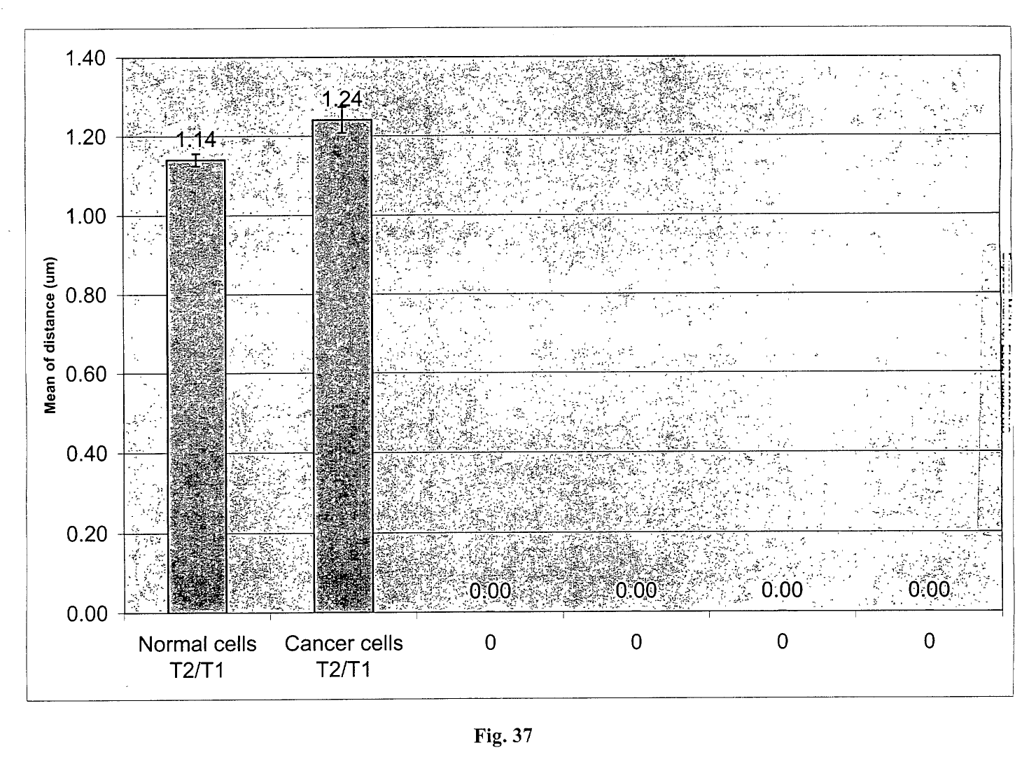

FIGS. 27 and 28 show a exemplary view of various possible embodiments for massively parallel system. In FIG. 37, an array 350 of multiple channels 352 are disposed parallel to one another. The array 350 may be a two dimensional array, as is shown in solid in FIG. 37, or alternatively, the array 350 may be three-dimensional, as is shown in dashed lines in FIG. 37. The array 350 of channels 352 are connected to a common inlet 354 as well as a common outlet 356. The three detecting positions t₁, t₂, and t₃ are also shown in FIG. 37. An optophoretic gradient 358 is disposed between the second and third detecting positions so as to slow down the particles or cells differentially based on their properties. If the array 350 is two-dimensional, the optophoretic gradient 358 preferably is formed as a line as is shown in FIG. 37. If, however, the array 350 is a three-dimensional array, the optophoretic gradient 358 preferably forms a plane (not shown) that passes through the channels 352. In the three-dimensional array 350, a detector system capable of determining which “layer” of the array the cell or particle is present is needed. One example is the electrode-based detector system illustrated herein in FIGS. 8A and 8B.

FIG. 28 discloses a three-dimensional parallel system that includes a series of stacked two-dimensional arrays 360 separated by a distance D. In this embodiment a light source 362 such as a Bessel beam, which is capable of reconstructing itself, is used to illuminate the arrays 360. FIG. 28 also shows an optophoretic gradient 364 disposed across the channels contained in each array 360.

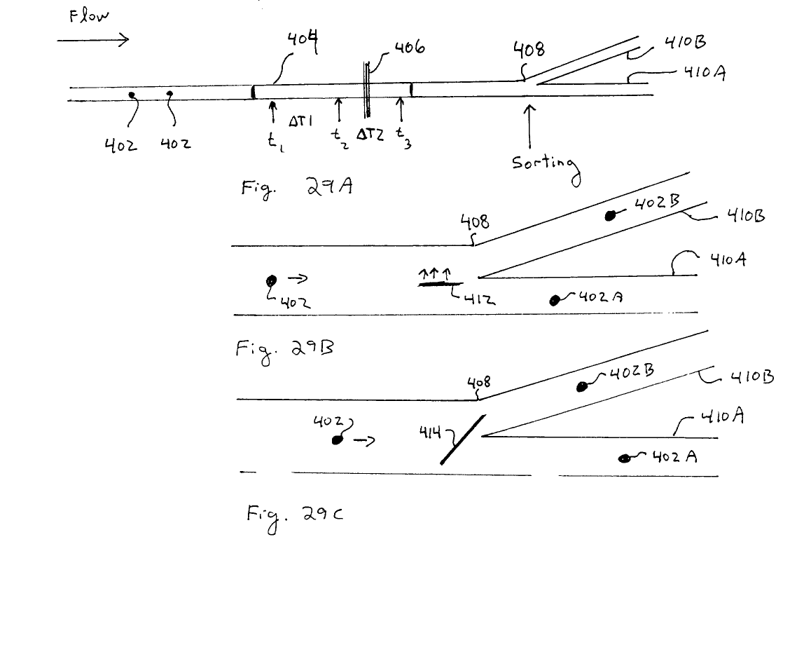

FIGS. 29A, 29B, and 29C illustrate an embodiment of a device 400 is capable of both characterizing and sorting cells or particles 402. In this embodiment a channel 404 is provided that contains a region having a plurality of detecting positions. FIG. 29A shows three such detecting positions t₁, t₂, and t₃. An Optophoretic gradient 406 is disposed between detecting positions t₂ and t₃. These detecting positions have one or more associated detectors (not shown) that are used to calculate the time-of-flight for region T1 and region T2. A comparison of the time-of-flight for each region can then be made to characterize the cell or particle 402. The device 400 also is capable of sorting cells or particles 402. The sorting step is carried out at a fork region 408 in which the channel 404 branches into two or more branches 410. FIG. 29A shows two such branches 410A and 410B, however, it should be understood that the single channel 404 may branch into any number of branches 410.

FIG. 29B illustrates one method used to sort the cells or particles 402. In this method a moving optical gradient 412 is used to sweep certain cells or particles 402 having certain desired characteristics into one of the braches 410. The optical gradient 412 starts at the one edge of the channel 404 (the lower edge shown in FIG. 29B) and scans generally perpendicular to the direction of fluid flow as shown by the arrows in FIG. 29B. When the cell or particle 402 encounters the moving optical gradient 412, the cell or particle 402 either passes into branch 410A or branch 410B. Those cells or particles 402 that interact more with the moving optical gradient 412 tend to be swept into the upper branch 410B (cell or particle 402B shown in FIG. 29B) while the other cells or particles 402 tend to pass into branch 410A (cell or particle 402A shown in FIG. 29B).

FIG. 29C illustrates an alternative method used to sort the cells or particles 402. In contrast to the prior embodiment, this embodiment employs a stationary optical gradient 414. The stationary optical gradient 414 is oriented at an angle to the direction of fluid flow. Those cells or particles 402 that interact more with the stationary optical gradient 414 tend to travel along the angled optical gradient and pass into the upper branch 410B (cell or particle 402B shown in FIG. 29C) while the other cells or particles 402 tend to pass into branch 410A (cell or particle 402A shown in FIG. 29C).

Experimental Data

A. Infection of Red Blood Cells with Plasmodium falciparum (malaria)

In one experiment, line scan as well as time-of-flight analysis was performed on cells to experimentally diagnose infection of red blood cells (RBCs) caused by Plasmodium falciparum, the parasite that causes malaria.

A variety of methods have been traditionally used to diagnose malaria infection. One method uses a visible stain such as Giemsa stain and subsequent microscopic evaluation. Alternatively, infection may be detected using nucleic-acid binding stains such as, for example, the fluorescent stain acridine orange followed by fluorescence microscopy or flow cytometric analysis. Still other diagnostic techniques use immunological methods that can detect the presence of Plasmodium-specific antigens. All of these methods, however, have limitations. All of the methods require the addition of exogenous reagents. Some of the methods kill the cells, thereby destroying sample integrity. Moreover, these methods often require expensive equipment that needs to be manned by skilled operators.

As an alternative to these diagnostic methods, Optophoretic interrogation of RBCs has been experimentally used to diagnose infection by Plasmodium falciparum. Plasmodium falciparum-infected RBC cell cultures and non-infected cell cultures were maintained in RPMI medium supplemented with HEPES buffer, NaHCO₃ and gentamicin. Prior to Optophoretic interrogation, cells were washed and diluted in phosphate-buffered saline containing 1% w/v bovine serum albumin (PBS/BSA).

Cells were subject to Optophoretic interrogation either in an unstained or stained condition. The stained cells were stained with the fluorescent nucleic-acid binding dye SybrGreen as a confirmatory test to distinguish infected RBCs (fluorescent) from non-infected RBCs (non-fluorescent). Some experiments were conducted using non-synchronized infection state cultures while other tests were conducted using synchronized cultures. Non-synchronized cultures comprised parasites that are at all stages of their life cycle. In contrast, synchronized cultures contained parasites that were at approximately the same stage of their infection cycle. Still other experiments were performed with varying levels of cellular infection. Because the culture used to grow the malaria parasite was self-limiting due to the presence of breakdown products of RBCs, no more than about 15% of the cells were infected. Higher infection rates with enriched populations of cells were achieved through the Percoll centrifugation-based method. Optophoretic interrogation was performed using line scan analysis as well as time-of-flight (TOF) analysis.

The EV data was generated using non-enriched, non-synchronized samples. The TOF data was generated using enriched, synchronized samples. Escape velocity measurements were taken using optical system similar to that shown in FIG. 20. FIG. 20 shows an optical system having illumination of a sample plate 194 from the top side and imaging from the bottom side. A laser 180 generates a first beam 181 which optionally passes through a spatial filter 182. The spatial filter 182 as shown includes lens 183 and aperture 184. The output of the spatial filter 182 is directed to a mirror 185 and passes through the objective 186 and is imaged onto the sample plate 187. The sample plate 187 and material supported on it may be imaged via an objective 188. An optional mirror 189 directs radiation to an optional filter 190 through an imaging lens 191 onto the detector 192. The detector 192 is coupled to an imaging system 193. Preferably, the imaging system 193 provides information to a control system 194 which controls various optical components of the system.

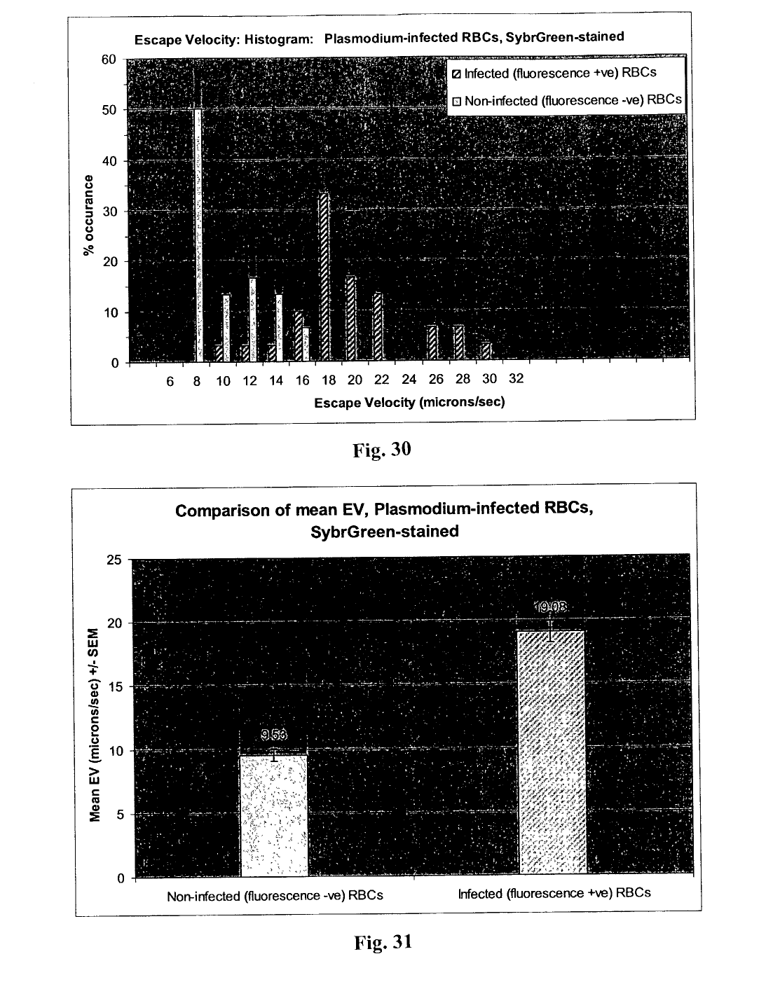

In this particular experimental setup, the laser was run at 100 mW power. FIG. 30 shows a histogram of the measured escape velocities of both infected and non-infected RBCs. In the tested sample, about 5% of the cells were infected with the parasite. The measured escape velocities for the non-infected RBCs were significantly lower than the escape velocities of the infected RBCs. FIG. 31 shows a comparison of the mean escape velocity for the infected and non-infected RBCs.

Time-of-flight measurements were also performed on a population of normal RBCs and a non-synchronized, mixed population of infected RBCs. With respect to the infected population, the population contained in excess of 70% infected cells. Time-of-flight measurements were made using a 2.6 W laser focused with a cylindrical lens. FIG. 32 shows a histogram of the time-of-flight measurements for normal RBCs and infected RBCs. As seen in FIG. 32, the infected RBCs show a slight shift upward in time-of-flight (TOF) values as compared to the non-infected (control) RBCs. FIG. 33 shows the comparison of the mean TOF values for the infected and control cells.

FIGS. 34 and 35 illustrate the results of another time-of-flight experiment performed on infected and non-infected RBCs. The same experimental setup was used as in the prior experiment (i.e., 2.6 W laser with cylindrical lens). In this experiment, however, synchronized cells were tested. In addition, in this experiment, over 95% of the cells were infected. FIG. 34 shows a histogram of TOF data for the infected and non-infected cells. As seen in FIG. 34, the infected cells show a noticeable increase in TOF values as compared to their non-infected counterparts. FIG. 35 shows the mean TOF values for both the infected and non-infected populations.

B. Characterization of Normal and Cancerous Cells

Optophoretic interrogation using time-of-flight analysis has been used to distinguish cancer cells from normal cells for breast carcinoma and skin melanoma. In this regard, Optophoretic interrogation can be used as a diagnostic tool to determine whether cells show the Optophoretic characteristics of cancer cells or normal cells. In addition, the Optophoretic methods can also be used to detect whether the sample cells are primary or metastatic cells. These provide a relatively quick way of diagnosing whether a sample contains cancerous cells. The techniques advantageously may be used with relatively small sample sizes.

Experiments have been conducted on human breast carcinoma cells as well as human melanoma cells. Tumor cell lines were purchased from ATCC and, when available, their normal counterparts were matched from the same patient. Cells were grown in culture until the time of testing. Adherent cells were detached from culture flasks using trypsin and resuspended in buffer. Cells were then subject to Optophoretic interrogation.

Samples of matched cancerous and non-cancerous cells from breast tissue (HS578T and HS578BST) were tested using a time-of-flight system. FIG. 36 illustrates a histogram of the ratio of T₂/T₁ plotted against the percentage of cells. The cancerous cells (HS578T) exhibited a larger T₂/T₁ ratio as compared to the normal cells (HS578BST). FIG. 37 illustrates the mean T₂/T₁ ratio for the cancerous and non-cancerous cells.

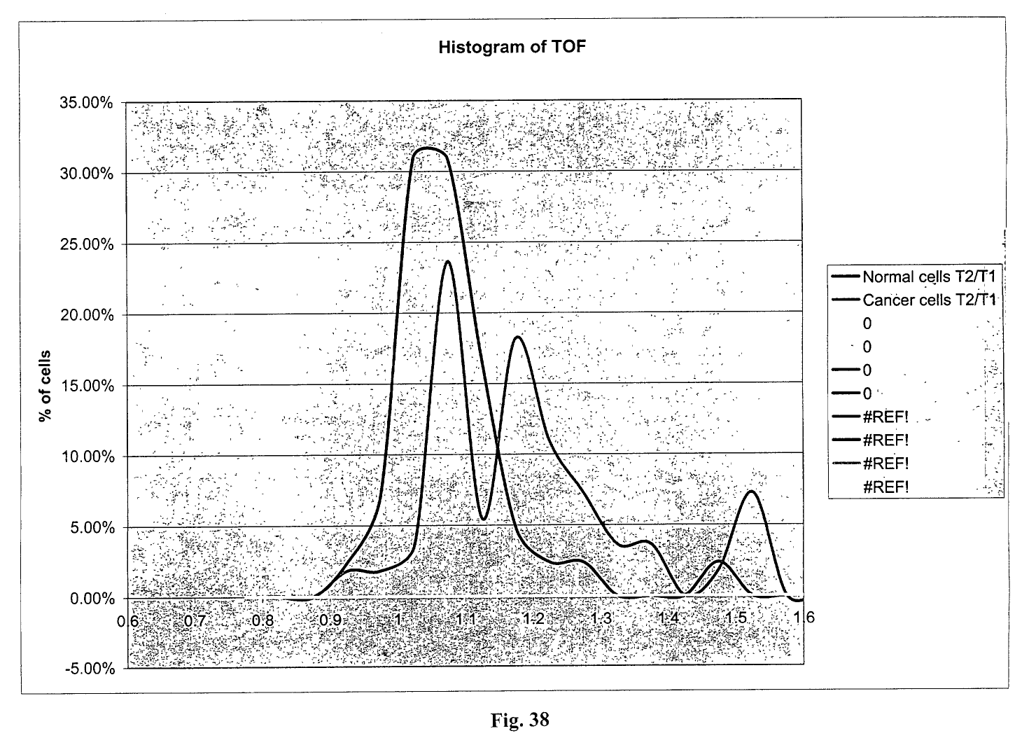

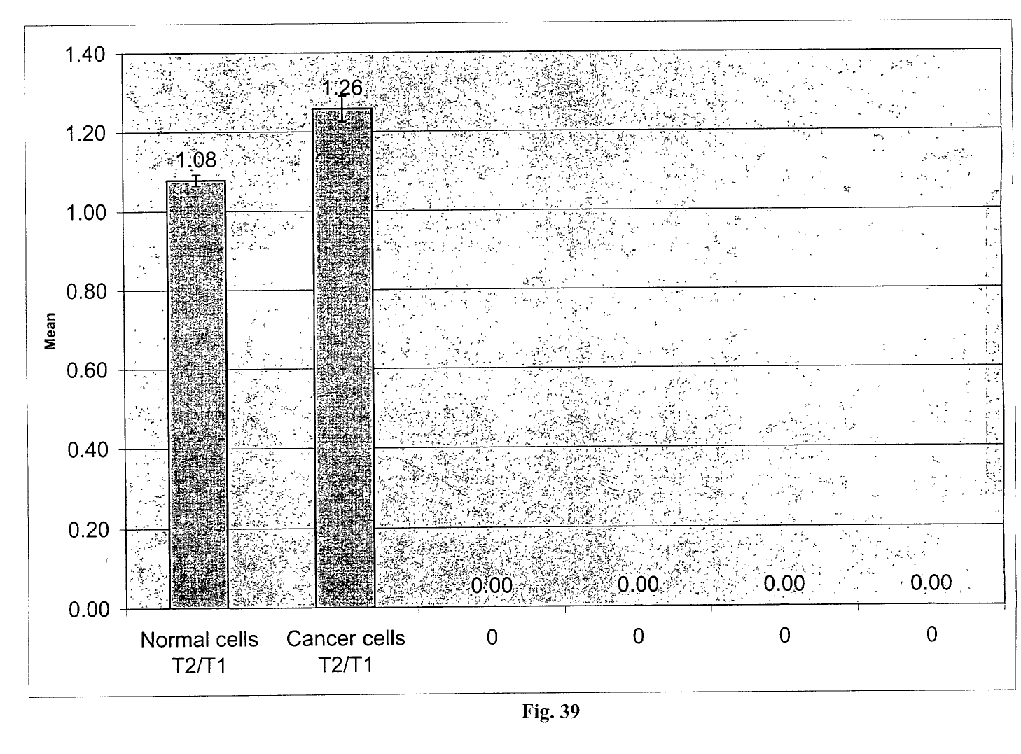

In yet another experiment, normal skin cells (CCD 1037) and malignant melanoma cells (WM 115) were subject to time-of-flight analysis. The time-of-flight analysis was performed using a laser powered at 2.6 W using a cylindrical lens . FIG. 38 illustrates the ratio of T₂/T₁ plotted against the percentage of cells. The cancerous cells (WM 115) exhibited a larger T₂/T₁ ratio as compared to the normal cells (CCD 1037). These results are consistent with the results seen in the time-of-flight data for breast carcinoma cells, namely, that the cancerous cells exhibit generally higher T₂/T₁ ratios. FIG. 39 illustrates the mean T₂/T₁ ratio for the cancerous and non-cancerous cells.

C. Characterization of Cells Using Beads

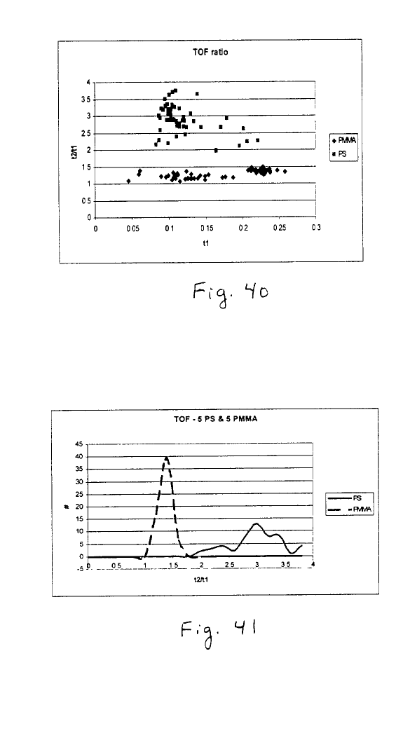

In this experiment, 5.1 μm polystyrene and 5.0 μm polymethylmethacrylate (PMMA) beads were subject to time-of-flight analysis on a diagnostic device of the type shown in FIGS. 32-34. These two samples of particles have different refractive indexes. The small difference in diameters was small enough that characterization was based on refractive index difference and not particle diameter. FIG. 40 illustrates a scatter plot of the time-of-flight (TOF) ratio as a function of t₁. FIG. 41 illustrates a histogram of the number of particles as a function of the TOF ratio t₂/t₁. As seen in FIGS. 40 and 41, the polystyrene beads have a larger TOF ratio as compared to the PMMA beads. The beads may be used as vehicles to carry different cells or populations of cells. Interrogation may then be performed on the underlying vehicle which, in turn, allows for the characterization and possible sorting of cells adhered to the surface of the beads. For example, beads made from polystyrene may have one or more compounds bound thereto that are specific to a particular cell or cell type. Other beads made from another material, i.e., PMMA, may include one or more different compounds thereon that bind to a different type of cell. Characterization and sorting may be performed using the Optophoretic differences in the carrier beads. In another related application, the beads may be used in an agglomeration assay in which beads have specific ligands attached to their surfaces. The ligand laden beads are then able to bind to cells having corresponding binding sites. In this type of assay, a single cell might be bound to multiple beads, each bead having a different ligand. These bead-ligand-cell complexes may then be analyzed optophoretically to analyze and differentiate the cells of interest.

D. Characterization of Wild Type and Mutant Yeast Strains

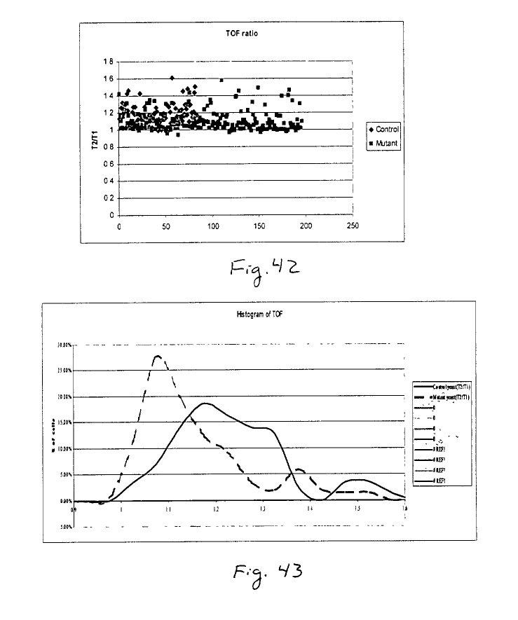

In this experiment, two strains of yeast, 24657 rho+ (wild type) and MYA-1133 rho(0) (mutant) were subject to time-of-flight testing on a diagnostic device of the type shown in FIGS. 32-34. The difference between the wild type and the mutant yeast strain is that the rho(0) strain lacks mitochondrial DNA. FIG. 42 illustrates a scatter plot of the time-of-flight (TOF) ratio as a function of the event number for the mutant and wild type cells. FIG. 43 illustrates a histogram of the percentage of yeast cells as a function of the TOF ratio t₂/t₁. As seen in FIGS. 42 and 43, the mutant strain rho(0) generally has a lower TOF ratio as compared to the wild type strain.

E. Characterization of HL60 Cells In Response to Treatment with DMSO

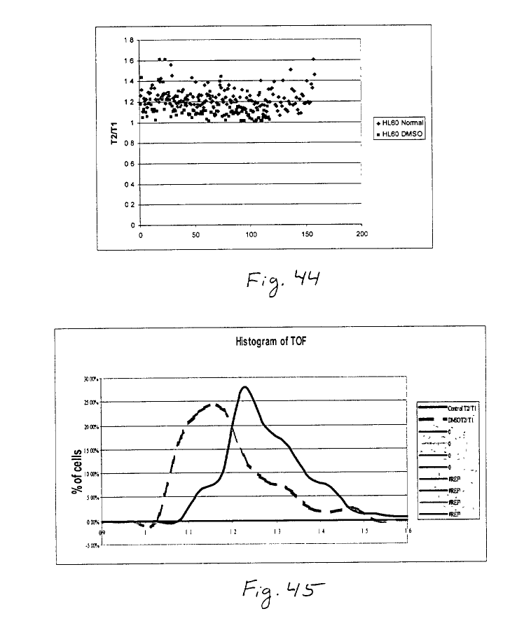

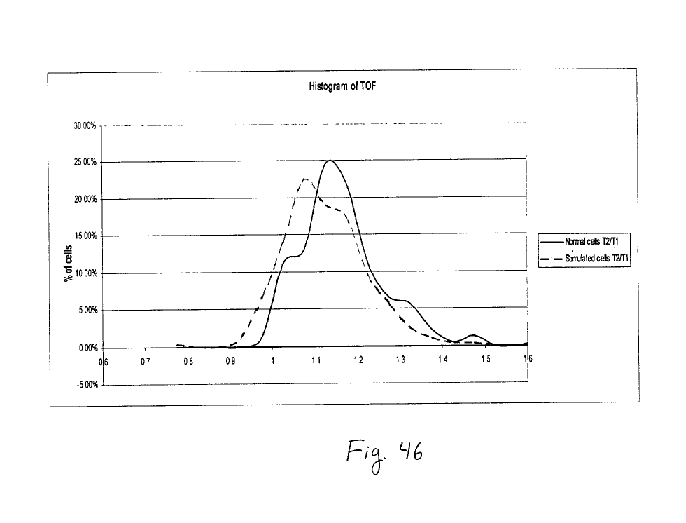

In this experiment, HL60 cells were treated with 1% dimethyl sulfoxide (DMSO) and subject to time-of-flight testing on a diagnostic device of the type shown in FIGS. 32-34. As a control, a set of HL60 cells that were not treated with DMSO were also subject to time-of-flight testing. Measurements were made after 40 hours of treatment with DMSO. DMSO is a known cell differentiation inducer. FIG. 44 illustrates a scatter plot of the time-of-flight (TOF) ratio as a function of the event number for the treated and non-treated HL60 cells. FIG. 45 illustrates a histogram of the percentage of cells as a function of the TOF ratio t₂/t₁. As seen in FIGS. 44 and 45, the HL60 cells treated with DMSO generally had a lower TOF ratio as compared to the non-treated HL60 cells.

F. Characterization of Activated and Unactivated T Cells

In this experiment, activated and unactivated T cells were subject to time-of-flight testing on a diagnostic device of the type shown in FIGS. 32-34. Phorbol mystirate acetate (PMA) and ionomycin were used to activate the T cells. T cells were cultured at a cell density on the order of 10⁻⁶ cells per ml in the presence of 0.5 μml PMA and 50 ng/ml ionomycin overnight in a CO₂ incubator. FIG. 46 illustrates a histogram of the percentage of cells as a function of the TOF ratio t₂/t₁. As seen in FIG. 46, the stimulated T cells generally had a lower TOF ratio as compared to the unstimulated T cells.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

We claim:

1. A device for characterizing cells or particles comprising:
a channel having an inlet and an outlet,
a source of fluid for flowing through the channel from the inlet to the outlet, the source of fluid carrying at least one cell or particle,
detectors for detecting the position of the at least one cell or particle within the channel at at least three points in time,
a light source for defining an optical gradient across at least a portion of the channel in a direction generally orthogonal to the fluid flow, and
an analysis system coupled to the detectors to characterize the at least one cell or particle.

2. The device according to claim 1, wherein the channel is defined in a substrate.

3. The device according to claim 1, wherein the device further includes an inlet reservoir.

4. The device according to claim 1, wherein the device further includes an outlet reservoir.

5. The device according to claim 1, wherein the detectors are discrete detectors.

6. The device according to claim 1, wherein the detectors are integrated detectors.

7. The device according to claim 1, further comprising an illumination system having a light source for illuminating a portion of the channel.

8. The device according to claim 7, wherein the illumination system comprises a pattern generator.

9. The device according to claim 7, wherein the illumination system comprises a scanning device.

10. The device according to claim 7, further comprising a detector mask.

11. The device according to claim 1, wherein the analysis system includes a display.

12. The device according to claim 1, further comprising a sorting system.

13. The device according to claim 1, wherein the analysis system controls the sorting system.

14. A device for characterizing a cell or particle comprising:
a channel having an inlet and an outlet, the channel containing a moving fluid therein for carrying the cell or particle from the inlet to the outlet;
a detector for detecting the presence of a cell or particle along a portion of the channel, the detector including a first detecting position, a second detecting position located downstream of the first detecting position, and a third detecting position located downstream of the second detecting position;
a light source providing an optical gradient disposed within the channel and between the second and third detection positions of the detector;
a control system coupled to the detector to receive and process detected signals from the detector.

15. The device according to claim 14, wherein the detector comprises a plurality of discrete detectors.

16. The device according to claim 14, wherein the detector comprises an integrated detector.

17. The device according to claim 14, wherein the light source comprises a laser for generating the optical gradient.

18. The device according to claim 14, further comprising an illumination system having a light source for illuminating a portion of the channel.

19. The device according to claim 18, wherein the illumination system comprises a pattern generator.

20. The device according to claim 18, wherein the illumination system comprises a scanning device.

21. The device according to claim 18, further comprising a detector mask.

22. The device according to claim 14, wherein the control system includes a display.

23. The device according to claim 14, wherein the distance between the first and third detecting positions is less than 200 microns.

24. The device according to claim 14, wherein the distance between the first and second detecting positions is equal to the distance between the second and third detecting positions.

25. The device according to claim 14, wherein the moving fluid has a substantially constant flow rate.

26. The device according to claim 22, wherein a timing diagram is displayable on the display.

27. The device according to claim 14, wherein the flow rate of the moving fluid is adjustable.

28. The device according to claim 14, wherein the flow rate of the moving fluid exceeds the escape velocity of the cell or particle.

29. A method for characterizing a cell or particle comprising the steps of:
flowing a cell or particle past first and second points defining a first zone;
measuring the time it takes the cell or particle to pass between the first and second points in the first zone;
flowing a cell or particle past first and second points defining a second zone;
subjecting the cell or particle to an optical gradient positioned in the second zone;
measuring the time it takes the cell or particle to pass between the first and second points in the second zone; and
comparing the measured times for the first and second zones for characterizing the cell or particle.

30. The method according to claim 29, further comprising the step of sorting the cell or particle based on the measured times for the first and second zones.

31. The method according to claim 29, wherein the second point in the first zone is also the first point in the second zone.

32. A method of determining a biological property of a cell or population of cells comprising the steps of:
flowing a cell past first and second points defining a first zone;
measuring the time it takes the cell to pass between the first and second points in the first zone;
flowing the cell past first and second points defining a second zone;
subjecting the cell to an optical gradient positioned in the second zone;
measuring the time it takes the cell to pass between the first and second points in the second zone; and
comparing the measured times for the first and second zones for the cell so as to determine a biological property of the cell based at least in part on the comparison.

33. The method according to claim 32, wherein the biological property comprises whether the cell is infected with an infectious agent.

34. The method according to claim 32, wherein the biological property comprises whether the cell is cancerous.

35. The method according to claim 34, wherein the biological property comprises the metastatic potential of the cell.

36. The method according to claim 32, wherein the biological property comprises detecting a phenotype change in the cell.

37. The method according to claim 32, wherein the biological property comprises detecting whether the cell is wild type or mutant.

38. The method according to claim 32, wherein the cell is a T cell.

39. The method according to claim 38, wherein the biological property comprises the activation level of the T cell.

40. A method of diagnosing a diseased state of one or more cells in a sample containing a plurality of cells comprising the steps of:
flowing the sample of cells through a first detecting region;
measuring the time it takes the cells to pass through the first detecting region;
flowing the cells through a second detecting region located downstream of the first detecting region;
subjecting the cells to an optical gradient positioned in the second detecting region;
measuring the time it takes the cells pass through the second detecting region; and
comparing the measured times for the first and second detecting regions for characterizing at least a portion of the cells in the sample as being in a diseased state or in a normal state.

41. The method according to claim 40, wherein the diseased state is cancer.

42. The method according to claim 40, wherein the diseased state is infection.

43. A method of analysis of an environmental sample containing a plurality of particles comprising the steps of:
flowing the sample of particles through a first detecting region;
measuring the time it takes the particles to pass through the first detecting region;
flowing the particles through a second detecting region located downstream of the first detecting region;
subjecting the particles to an optical gradient positioned in the second detecting region;
measuring the time it takes the particles pass through the second detecting region; and
comparing the measured times for the first and second detecting regions for characterizing at least a portion of the particles in the sample.

44. The method according to claim 43, wherein the sample is an airborne sample.

45. The method according to claim 43, wherein the sample is a waterborne sample.

46. A device for characterizing cells or particles comprising:
at least one channel having an inlet and an outlet;
a source of fluid for flowing through the at least one channel from the inlet to the outlet, the source of fluid carrying cells or particles;
a first light source for defining a detection beam within the at least one channel, the detection beam being disposed in the channel and generally parallel to the direction of fluid flow;
a first detector for detecting the presence of a cell or particle along a portion of the at least one channel;
a second detector for detecting the presence of a cell or particle along another portion of the at least one channel, the second detector including a first detecting position, a second detecting position located downstream of the first detection position, and a third detecting position located downstream of the second detecting position;
a second light source for providing an optical gradient disposed within the at least one channel and between the second and third detection positions of the second detector; and
a control system coupled to the first and second detectors to receive and process detected signals from the first and second detectors.

47. The device according to claim 46, the first light source comprising a coherent light source.

48. The device according to claim 46, the second light source comprising a coherent light source.

49. The device according to claim 46, further comprising a flow pump coupled to one of the inlet and outlet of the channel.

50. The device according to claim 46, wherein the channel is disposed inside a microfluidic mounting system.

51. The device according to claim 46, further comprising an external computer coupled to the device via a computer interface.

52. The device according to claim 46, further comprising a plurality of channels.
